(19) United States
(12) Patent Application Publication
(10) Pub. No.: US 2024/0024477 A1
(43) Pub. Date: Jan. 25, 2024

(54) T CELL RECEPTORS AND USES THEREOF

(71) Applicant: Oxford University Innovation Limited, Oxford (Oxfordshire) (GB)

(72) Inventors: Honbing YANG, Oxford (Headington) (GB); Geraldine GILLESPIE, Oxford (Headington) (GB); Margarida REI, Oxford (Headington) (GB); Simon BRACKENRIDGE, Oxford (Headington) (GB); Andrew MCMICHAEL, Oxford (Headington) (GB); Hong SUN, Oxford (Headington) (GB)

(21) Appl. No.: 18/038,722

(22) PCT Filed: Dec. 2, 2021
(86) PCT No.: PCT/GB2021/053155

§ 371 Date: May 25, 2023

(30) Foreign Application Priority Data
Dec. 2, 2020 (GB) 2019019.5

(51) Int. Cl. A61K 39/00; C07K 14/725; C07K 16/10; A61P 31/18; A61K 38/20; A61K 38/21; A61K 39/395

(52) U.S. Cl. CPC A61K 39/4632; C07K 14/7051; C07K 16/1045; A61K 39/4611; A61P 31/18; A61K 38/2013; A61K 38/217; A61K 39/3955

(57) ABSTRACT

The invention relates to a T cell receptor (TCR) which is HIV-1 specific and HLA-E restricted. Particularly, the TCR is capable of binding to a peptide of RMYSPTSIL or a peptide of RMYSPTSIL in complex with HLA-E, or a peptide of ILVESPAVL or a peptide of ILVESPAVL in complex with HLA-E. The invention also relates to a nucleic acids and vector encoding the TCR.

### TECHNICAL FIELD

The present invention relates to T cell receptors (TCRs) which are HIV-1 specific and HLA-E restricted, and to nucleic acids and vectors encoding the TCRs. The invention also relates to the use of TCR receptors to treat and/or prevent HIV-1 and/or AIDS.

### BACKGROUND OF THE INVENTION

T cell receptors (TCRs) are naturally expressed by CD4+ and CD8+ T cells. TCRs can recognise short peptide antigens that are displayed on the surface of antigen presenting cells in complex with Major Histocompatibility Complex (MHC) molecules (in humans, MHC molecules are also known as Human Leukocyte Antigens, or HLA). CD8+ T cells, which are also termed cytotoxic T cells, have TCRs that specifically recognize peptides bound to MHC class I molecules. CD8+ T cells are generally responsible for finding and mediating the destruction of infected or diseased cells, including cancerous and virally infected cells.

TCR sequences are generally described with reference to IMGT (LeFranc and LeFranc, (2001). “T cell Receptor Factsbook”, Academic Press; Lefranc, (2011), Cold Spring Harb Protoc 2011(6):595-603; Lefranc, (2001), Curr Protoc Immunol Appendix 1: Appendix 10; and Lefranc, (2003), Leukemia 17(1):260-266), which are widely known and accessible to those working in the TCR field.

Briefly, αβ TCRs consist of two disulphide linked chains. Each chain (alpha and beta) is generally regarded as comprising two domains, namely variable and constant domains. The alpha variable domain comprises a variable segment and joining segment. The beta variable domain also comprises a variable and joining segment, and also usually contains one or more short diversity segments between the variable and joining segments, which is usually regarded as forming part of the joining segment). The term “alpha chain variable domain” therefore refers to the concatenation of TRAV and TRAJ segments, and the term “alpha chain constant domain” refers to the TRAC region. Likewise the term “beta chain variable domain” refers to the concatenation of TRBV and TRBD/TRBJ segments, and the term “beta chain constant domain refers to the TRBC region.

The variable domain of each chain is located N-terminally and comprises three Complementarity Determining Regions (CDRs) embedded in a framework sequence (FR). The CDRs comprise the recognition site for peptide-MHC binding. There are multiple genes coding for alpha chain variable (Vα) segments and several genes coding for beta chain variable (Vβ) segments, which are distinguished by their framework, CDR1 and CDR2 sequences, and by a partly defined CDR3 sequence. The Vα and Vβ genes are referred to in IMGT nomenclature by the prefix TRAV and TRBV, which encode variable segments. Likewise there are several joining or J genes, termed TRAJ or TRBJ, for the alpha and beta chain respectively, which encode joining segments, and for the beta chain, diversity or D genes termed TRBD which encode diversity segments. The large diversity of T cell receptor chains results from combinatorial rearrangements between the various V and J alpha genes and the V, J and D beta genes, which include allelic variants, together with some random junctional diversity. The constant, or C, regions of TCR alpha and beta chains are referred to as TRAC and TRBC respectively.

Human immunodeficiency virus (HIV) infection and related diseases are a major public health problem worldwide, with approximately 38 million people globally living with the virus in 2018. The virus is an enveloped retrovirus belonging to the lentivirus group, which infects and destroys CD4+ T-cells of the immune system. This can lead to the development of acquired immunodeficiency syndrome (AIDS), in which the immune system is weakened to the extent that secondary infections can easily establish, eventually causing fatality.

Current treatments rely on the use of combination antiretroviral therapy (ART) to control viral infection. However, these treatments are unable to completely eradicate infection due to the stable integration of viral genes into host cell chromosomes, leading to the rapid establishment of a reservoir of long-lived, latently infected CD4+ T cells. Additionally, ART requires strict adherence to treatment regimens and is associated with lifelong costs and potential toxicity effects.

An important part of the natural response to virus-infected cells is that of CD8+ cytotoxic T-cells, which recognise and destroy HIV-1 infected cells. The T cells recognize short peptide sequences bound to HLA-A, -B or -C molecules on the surface of infected cells. HLA-A, -B and -C molecules are highly genetically polymorphic and each HLA type presents different peptides. Recapitulating and amplifying the mechanisms underlying the CD8+ T cell response to the virus, represents a promising approach to eradicating virus repertoires.

Vaccines designed to stimulate the conventional T cell responses have been developed and used in combination with viral reactivation reagents, aiming to eradicate HIV reservoirs; however, to date these have proved largely ineffective (Schooley et al. 2012, J Infect Dis 202,705; Casazza et al. 2013, J. Infect Dis 207, 1829).

Because of the genetic diversity of HLA-A, -B and -C, no widely applicable therapeutic anti- HIV-1 TCR sequences are known in the art, and methods for the identification of TCRs with specificity characteristics amenable to general therapeutic use is technically burdensome. The skilled person would need to identify several TCRs to cover more than half the population.

There is therefore a need in the art to identify new or alternative TCRs which can reduce or prevent HIV-1 infection.

### SUMMARY OF INVENTION

In a first aspect, there is provided a polypeptide encoding a T-cell receptor (TCR), wherein the TCR is virus specific and HLA-E restricted. Such an HLA-E restricted T-cell offers the solution to the problem of genetic diversity experienced in classical MHCI restricted TCRs, because HLA-E is nonpolymorphic; there are only two common alleles in humans, which differ by only one amino acid which has no impact on peptide binding. Thus, HLA-E restricted HIV-1 specific TCRs and T-cells comprising such TCRs can be used in all infected patients to prevent and/or reduce viral replication and therefore treat or prevent infection with viruses such as HIV-1.

The virus may be HIV. The HIV may be HIV-1 or HIV-2.

The TCR may be capable of binding to a peptide of RMYSPTSIL (SEQ ID NO: 1) or a peptide of SEQ ID NO: 1 in complex with HLA-E. The TCR may be capable of binding to a peptide of ILVESPAVL (SEQ ID NO: 110) or a peptide of SEQ ID NO: 110 in complex with HLA-E

The TCR may comprise an alpha chain variable domain and a beta chain variable domain comprising:
a) a CDR3-alpha of CAFMKLHSGAGSYQLTF (SEQ ID NO: 2) or a CDR3-alpha with at least 90% identity, such as 90%, 95%, 96%, 97%, 98%, 99% of 100% identity to SEQ ID NO: 2, and a CDR3-beta of CASSLWAVGYGYTF (SEQ ID NO: 3) or a CDR3-beta with at least 90% identity, such as 90%, 95%, 96%, 97%, 98%, 99% of 100% identity to SEQ ID NO: 3; or
b) a CDR3-alpha of CAFDNNNDMRF (SEQ ID NO: 4) or a CDR3-alpha with at least 90% identity, such as 90%, 95%, 96%, 97%, 98%, 99% of 100% identity to SEQ ID NO: 4, and a CDR3-beta of CASSLVGAITEAFF (SEQ ID NO: 5) or a CDR3-beta with at least 90% identity, such as 90%, 95%, 96%, 97%, 98%, 99% of 100% identity to SEQ ID NO: 5; or
c) a CDR3-alpha of CAFVDGAGSYQLTF (SEQ ID NO: 6) or a CDR3-alpha with at least 90% identity, such as 90%, 95%, 96%, 97%, 98%, 99% of 100% identity to SEQ ID NO: 6, and a CDR3-beta of CASSVGNSNSPLHF (SEQ ID NO: 7) or a CDR3-beta with at least 90% identity, such as 90%, 95%, 96%, 97%, 98%, 99% of 100% identity to SEQ ID NO: 7; or
d) a CDR3-alpha of CAASGLFIQGGSEKLVF (SEQ ID NO: 8) or a CDR3-alpha with at least 90% identity, such as 90%, 95%, 96%, 97%, 98%, 99% of 100% identity to SEQ ID NO: 8, and a CDR3-beta of CASSVGNSNSPLHF (SEQ ID NO: 9) or a CDR3-beta with at least 90% identity, such as 90%, 95%, 96%, 97%, 98%, 99% of 100% identity to SEQ ID NO: 9; or
e) a CDR3-alpha of CAVYGSGKLTF (SEQ ID NO: 10) or a CDR3-alpha with at least 90% identity, such as 90%, 95%, 96%, 97%, 98%, 99% of 100% identity to SEQ ID NO: 10, and a CDR3-beta of CASSFGPSSGANVLTF (SEQ ID NO: 11) or a CDR3-beta with at least 90% identity, such as 90%, 95%, 96%, 97%, 98%, 99% of 100% identity to SEQ ID NO: 11; or
f) a CDR3-alpha of CAFTLYSGGGADGLTF (SEQ ID NO: 12) or a CDR3-alpha with at least 90% identity, such as 90%, 95%, 96%, 97%, 98%, 99% of 100% identity to SEQ ID NO: 12, and a CDR3-beta of CASSLPTSLSTDTQYF (SEQ ID NO: 13) or a CDR3-beta with at least 90% identity, such as 90%, 95%, 96%, 97%, 98%, 99% of 100% identity to SEQ ID NO: 13; or
g) a CDR3-alpha of CAMSWNSGNTPLVF (SEQ ID NO: 14) or a CDR3-alpha with at least 90% identity, such as 90%, 95%, 96%, 97%, 98%, 99% of 100% identity to SEQ ID NO: 14, and a CDR3-beta of CASSVTGVRNTIYF (SEQ ID NO: 15) or a CDR3-beta with at least 90% identity, such as 90%, 95%, 96%, 97%, 98%, 99% of 100% identity to SEQ ID NO: 15; or
h) a CDR3-alpha of CAAYGQKLLF (SEQ ID NO: 16) or a CDR3-alpha with at least 90% identity, such as 90%, 95%, 96%, 97%, 98%, 99% of 100% identity to SEQ ID NO: 16, and a CDR3-beta of CASSLLEPDLNTGELFF (SEQ ID NO: 17) or a CDR3-beta with at least 90% identity, such as 90%, 95%, 96%, 97%, 98%, 99% of 100% identity to SEQ ID NO: 17; or
i) a CDR3-alpha of CAVNTDKLIF (SEQ ID NO: 18) or a CDR3-alpha with at least 90% identity, such as 90%, 95%, 96%, 97%, 98%, 99% of 100% identity to SEQ ID NO: 18, and a CDR3-beta of CASSNPGNSDF (SEQ ID NO: 19) or a CDR3-beta with at least 90% identity, such as 90%, 95%, 96%, 97%, 98%, 99% of 100% identity to SEQ ID NO: 19; or
j) a CDR3-alpha of CAAVSTGSARQLTF (SEQ ID NO: 20) or a CDR3-alpha with at least 90% identity, such as 90%, 95%, 96%, 97%, 98%, 99% of 100% identity to SEQ ID NO: 20, and a CDR3-beta of CASSLAKGANVLTF (SEQ ID NO: 21) or a CDR3-beta with at least 90% identity, such as 90%, 95%, 96%, 97%, 98%, 99% of 100% identity to SEQ ID NO: 21; or
k) a CDR3-alpha of CVVSAWDPAAGNKLTF (SEQ ID NO: 22) or a CDR3-alpha with at least 90% identity, such as 90%, 95%, 96%, 97%, 98%, 99% of 100% identity to SEQ ID NO: 22, and a CDR3-beta of CASSPGGQGLDTQYF (SEQ ID NO: 23) or a CDR3-beta with at least 90% identity, such as 90%, 95%, 96%, 97%, 98%, 99% of 100% identity to SEQ ID NO: 23; or
l) a CDR3-alpha of CAYNRNDMRF (SEQ ID NO: 24) or a CDR3-alpha with at least 90% identity, such as 90%, 95%, 96%, 97%, 98%, 99% of 100% identity to SEQ ID NO: 24, and a CDR3-beta of CASSTDRDNQPQHF (SEQ ID NO: 25) or a CDR3-beta with at least 90% identity, such as 90%, 95%, 96%, 97%, 98%, 99% of 100% identity to SEQ ID NO: 25; or
m) a CDR3-alpha of CALSEALTSGTYKYIF (SEQ ID NO: 26) or a CDR3-alpha with at least 90% identity, such as 90%, 95%, 96%, 97%, 98%, 99% of 100% identity to SEQ ID NO: 26, and a CDR3-beta of CSASVGKSSYEQYV (SEQ ID NO: 27) or a CDR3-beta with at least 90% identity, such as 90%, 95%, 96%, 97%, 98%, 99% of 100% identity to SEQ ID NO: 27; or
n) a CDR3-alpha of CAVLLITQGGSEKLVF (SEQ ID NO: 111) or a CDR3-alpha with at least 90% identity, such as 90%, 95%, 96%, 97%, 98%, 99% of 100% identity to SEQ ID NO: 111, and a CDR3-beta of CASSVGGTNTQYF (SEQ ID NO: 112) or a CDR3-beta with at least 90% identity, such as 90%, 95%, 96%, 97%, 98%, 99% of 100% identity to SEQ ID NO: 112; or
o) a CDR3-alpha of CAVRDEDARLMF (SEQ ID NO: 113) or a CDR3-alpha with at least 90% identity, such as 90%, 95%, 96%, 97%, 98%, 99% of 100% identity to SEQ ID NO: 113, and a CDR3-beta of CSARGGGNRESHYGYTF (SEQ ID NO: 114) or a CDR3-beta with at least 90% identity, such as 90%, 95%, 96%, 97%, 98%, 99% of 100% identity to SEQ ID NO: 114; or
p) a CDR3-alpha of CAGQYSGGGADGLTF (SEQ ID NO: 115) or a CDR3-alpha with at least 90% identity, such as 90%, 95%, 96%, 97%, 98%, 99% of 100% identity to SEQ ID NO: 115, and a CDR3-beta of CASSLPDSSETQYF (SEQ ID NO: 116) or a CDR3-beta with at least 90% identity, such as 90%, 95%, 96%, 97%, 98%, 99% of 100% identity to SEQ ID NO: 116; or
q) a CDR3-alpha of CALSEYGGATNKLIF (SEQ ID NO: 117) or a CDR3-alpha with at least 90% identity, such as 90%, 95%, 96%, 97%, 98%, 99% of 100% identity to SEQ ID NO: 117, and a CDR3-beta of CATSRFLEGKDTEAFF (SEQ ID NO: 118) or a CDR3-beta with at least 90% identity, such as 90%, 95%, 96%, 97%, 98%, 99% of 100% identity to SEQ ID NO: 118; or
r) a CDR3-alpha of CAGGGGADGLTF (SEQ ID NO: 119) or a CDR3-alpha with at least 90% identity, such as 90%, 95%, 96%, 97%, 98%, 99% of 100% identity to SEQ ID NO: 119, and a CDR3-beta of CSVAETGTEAFF (SEQ ID NO: 120) or a CDR3-beta with at least 90% identity, such as 90%, 95%, 96%, 97%, 98%, 99% of 100% identity to SEQ ID NO: 120; or
s) a CDR3-alpha of CALSEAYSGAGSYQLTF (SEQ ID NO: 121) or a CDR3-alpha with at least 90% identity, such as 90%, 95%, 96%, 97%, 98%, 99% of 100% identity to SEQ ID NO: 121, and a CDR3-beta of CASNVGEGYNQPQHF (SEQ ID NO: 122) or a CDR3-beta with at least 90% identity, such as 90%, 95%, 96%, 97%, 98%, 99% of 100% identity to SEQ ID NO: 122; or
t) a CDR3-alpha of CAAWAPTNFGNEKLTF (SEQ ID NO: 123) or a CDR3-alpha with at least 90% identity, such as 90%, 95%, 96%, 97%, 98%, 99% of 100% identity to SEQ ID NO: 123, and a CDR3-beta of CASSVGYPGELFF (SEQ ID NO: 124) or a CDR3-beta with at least 90% identity, such as 90%, 95%, 96%, 97%, 98%, 99% of 100% identity to SEQ ID NO: 124; or
u) a CDR3-alpha of CAETLTHGGATNKLIF (SEQ ID NO: 125) or a CDR3-alpha with at least 90% identity, such as 90%, 95%, 96%, 97%, 98%, 99% of 100% identity to SEQ ID NO: 125, and a CDR3-beta of CASSEPGAAYEQYF (SEQ ID NO: 126) or a CDR3-beta with at least 90% identity, such as 90%, 95%, 96%, 97%, 98%, 99% of 100% identity to SEQ ID NO: 126; or
v) a CDR3-alpha of CKGGAQKLVF (SEQ ID NO: 127) or a CDR3-alpha with at least 90% identity, such as 90%, 95%, 96%, 97%, 98%, 99% of 100% identity to SEQ ID NO: 127, and a CDR3-beta of CASSLGRSYEQYF (SEQ ID NO: 128) or a CDR3-beta with at least 90% identity, such as 90%, 95%, 96%, 97%, 98%, 99% of 100% identity to SEQ ID NO: 128; or
w) a CDR3-alpha of CAVRDRNNFNKFYF (SEQ ID NO: 129) or a CDR3-alpha with at least 90% identity, such as 90%, 95%, 96%, 97%, 98%, 99% of 100% identity to SEQ ID NO: 129, and a CDR3-beta of CASSGVKGTGSEKLFF (SEQ ID NO: 130) or a CDR3-beta with at least 90% identity, such as 90%, 95%, 96%, 97%, 98%, 99% of 100% identity to SEQ ID NO: 130; or
x) a CDR3-alpha of CAMREGAGAGSYQLTF (SEQ ID NO: 131) or a CDR3-alpha with at least 90% identity, such as 90%, 95%, 96%, 97%, 98%, 99% of 100% identity to SEQ ID NO: 131, and a CDR3-beta of CASLAGQGRSEAFF (SEQ ID NO: 132) or a CDR3-beta with at least 90% identity, such as 90%, 95%, 96%, 97%, 98%, 99% of 100% identity to SEQ ID NO: 132; or
y) a CDR3-alpha of CAVSTGGGNKLTF (SEQ ID NO: 133) or a CDR3-alpha with at least 90% identity, such as 90%, 95%, 96%, 97%, 98%, 99% of 100% identity to SEQ ID NO: 133, and a CDR3-beta of CASSDGLRGSVRYEQYF (SEQ ID NO: 134) or a CDR3-beta with at least 90% identity, such as 90%, 95%, 96%, 97%, 98%, 99% of 100% identity to SEQ ID NO: 134; or
z) a CDR3-alpha of CAVVWATRLMF (SEQ ID NO: 135) or a CDR3-alpha with at least 90% identity, such as 90%, 95%, 96%, 97%, 98%, 99% of 100% identity to SEQ ID NO: 135, and a CDR3-beta of CAIGGQEILMHGYTF (SEQ ID NO: 136) or a CDR3-beta with at least 90% identity, such as 90%, 95%, 96%, 97%, 98%, 99% of 100% identity to SEQ ID NO: 136; or
aa) a CDR3-alpha of CAATFVSGSARQLTF (SEQ ID NO: 137) or a CDR3-alpha with at least 90% identity, such as 90%, 95%, 96%, 97%, 98%, 99% of 100% identity to SEQ ID NO: 137, and a CDR3-beta of CASSLRRAHTDTQYF (SEQ ID NO: 138) or a CDR3-beta with at least 90% identity, such as 90%, 95%, 96%, 97%, 98%, 99% of 100% identity to SEQ ID NO: 138; or
bb) a CDR3-alpha of CALSRRYSTLTF (SEQ ID NO: 139) or a CDR3-alpha with at least 90% identity, such as 90%, 95%, 96%, 97%, 98%, 99% of 100% identity to SEQ ID NO: 139, and a CDR3-beta of CASRLTDSYEQYF (SEQ ID NO: 140) or a CDR3-beta with at least 90% identity, such as 90%, 95%, 96%, 97%, 98%, 99% of 100% identity to SEQ ID NO: 140; or
cc) a CDR3-alpha of CAAGSSNTGKLIF (SEQ ID NO: 141) or a CDR3-alpha with at least 90% identity, such as 90%, 95%, 96%, 97%, 98%, 99% of 100% identity to SEQ ID NO: 141, and a CDR3-beta of CSVEFASKGYEQYF (SEQ ID NO: 142) or a CDR3-beta with at least 90% identity, such as 90%, 95%, 96%, 97%, 98%, 99% of 100% identity to SEQ ID NO: 142; or
dd) a CDR3-alpha of CAATPLLGADKIIF (SEQ ID NO: 143) or a CDR3-alpha with at least 90% identity, such as 90%, 95%, 96%, 97%, 98%, 99% of 100% identity to SEQ ID NO: 143, and a CDR3-beta of CASSFRGEAEAFF (SEQ ID NO: 144) or a CDR3-beta with at least 90% identity, such as 90%, 95%, 96%, 97%, 98%, 99% of 100% identity to SEQ ID NO: 144; or
ee) a CDR3-alpha of CAGSGYSGAGSYQLTF (SEQ ID NO: 145) or a CDR3-alpha with at least 90% identity, such as 90%, 95%, 96%, 97%, 98%, 99% of 100% identity to SEQ ID NO: 145, and a CDR3-beta of CASSFSAGTDTQYF (SEQ ID NO: 146) or a CDR3-beta with at least 90% identity, such as 90%, 95%, 96%, 97%, 98%, 99% of 100% identity to SEQ ID NO: 146; or
ff) a CDR3-alpha of CAVSSQVTGGSEKLVF (SEQ ID NO: 147) or a CDR3-alpha with at least 90% identity, such as 90%, 95%, 96%, 97%, 98%, 99% of 100% identity to SEQ ID NO: 147, and a CDR3-beta of CASSPPTLGYGYTF (SEQ ID NO: 148) or a CDR3-beta with at least 90% identity, such as 90%, 95%, 96%, 97%, 98%, 99% of 100% identity to SEQ ID NO: 148.

The TCR may comprise an alpha chain variable domain comprising a CDR1-alpha of TSENNY (SEQ ID NO: 28), a CDR2-alpha of QEAYKQQN (SEQ ID NO: 29) and a CDR3-alpha of SEQ ID NO: 2; and/or a beta alpha chain variable domain comprising a CDR1-beta of SGHDY (SEQ ID NO: 30), a CDR2-beta of FNNNVP (SEQ ID NO: 31) and a CDR3-beta of SEQ ID NO: 3.

The TCR may comprise an alpha chain variable domain comprising a CDR1-alpha of TSENNY (SEQ ID NO: 32), a CDR2-alpha of QEAYKQQN (SEQ ID NO: 33) and a CDR3-alpha of SEQ ID NO: 4; and/or a beta alpha chain variable domain comprising a CDR1-beta of SGHDT (SEQ ID NO: 34), a CDR2-beta of YYEEEE (SEQ ID NO: 35; and a CDR3-beta of SEQ ID NO: 5.

The TCR may comprise an alpha chain variable domain comprising a CDR1-alpha of TSENNY (SEQ ID NO: 36), a CDR2-alpha of QEAYKQQN (SEQ ID NO: 37) and a CDR3-alpha of SEQ ID NO: 6; and/or a beta alpha chain variable domain comprising a CDR1-beta of SGDLS (SEQ ID NO: 38), a CDR2-beta of YYNGEE (SEQ ID NO: 39) and a CDR3-beta of SEQ ID NO:7.

The TCR may comprise an alpha chain variable domain comprising a CDR1-alpha of NTAFDY (SEQ ID NO: 40), a CDR2-alpha of IRPDVSE (SEQ ID NO: 41) and a CDR3-alpha of SEQ ID NO: 8; and/or a beta alpha chain variable domain comprising a CDR1-beta of SGDLS (SEQ ID NO: 42), a CDR2-beta of YYNGEE (SEQ ID NO: 43) and a CDR3-beta of SEQ ID NO: 9.

The TCR may comprise an alpha chain variable domain comprising a CDR1-alpha of DRGSQS (SEQ ID NO: 44), a CDR2-alpha of IYSNGD (SEQ ID NO: 45) and a CDR3-alpha of SEQ ID NO: 10; and/or a beta alpha chain variable domain comprising a CDR1-beta of MDHEN (SEQ ID NO: 46), a CDR2-beta of SYDVKM (SEQ ID NO: 47) and a CDR3-beta of SEQ ID NO: 11.

The TCR may comprise an alpha chain variable domain comprising a CDR1-alpha of TSENNYY (SEQ ID NO: 48), a CDR2-alpha of QEAYKQQN (SEQ ID NO: 49) and a CDR3-alpha of SEQ ID NO: 12; and/or a beta alpha chain variable domain comprising a CDR1-beta of MDHEN (SEQ ID NO: 50), a CDR2-beta of SYDVKM (SEQ ID NO: 51) and a CDR3-beta of SEQ ID NO: 13.

The TCR may comprise an alpha chain variable domain comprising a CDR1-alpha of NSAFQY (SEQ ID NO: 52), a CDR2-alpha of TYSSGN (SEQ ID NO: 53) and a CDR3-alpha of SEQ ID NO: 14; and/or a beta alpha chain variable domain comprising a CDR1-beta of SNHLY (SEQ ID NO: 54), a CDR2-beta of FYNNEI (SEQ ID NO: 55) and a CDR3-beta of SEQ ID NO: 15.

The TCR may comprise an alpha chain variable domain comprising a CDR1-alpha of DSASNY (SEQ ID NO: 56), a CDR2-alpha of IRSNVGE (SEQ ID NO: 57) and a CDR3-alpha of SEQ ID NO: 16; and/or a beta alpha chain variable domain comprising a CDR1-beta of MDHEN (SEQ ID NO: 58), a CDR2-beta of SYDVKM (SEQ ID NO: 59) and a CDR3-beta of SEQ ID NO: 17.

The TCR may comprise an alpha chain variable domain comprising a CDR1-alpha of TSGFNG (SEQ ID NO: 60), a CDR2-alpha of NVLDGL (SEQ ID NO: 61) and a CDR3-alpha of SEQ ID NO: 18; and/or a beta alpha chain variable domain comprising a CDR1-beta of SEHNR (SEQ ID NO: 62), a CDR2-beta of FQNEAQ (SEQ ID NO: 63) and a CDR3-beta of SEQ ID NO: 19.

The TCR may comprise an alpha chain variable domain comprising a CDR1-alpha of NSMFDY (SEQ ID NO: 64), a CDR2-alpha of ISSIKDK (SEQ ID NO: 65) and a CDR3-alpha of SEQ ID NO: 20; and/or a beta alpha chain variable domain comprising a CDR1-beta of SGHRS (SEQ ID NO: 66), a CDR2-beta of YFSETQ (SEQ ID NO: 67) and a CDR3-beta of SEQ ID NO: 21.

The TCR may comprise an alpha chain variable domain comprising a CDR1-alpha of VSPFSN (SEQ ID NO: 68), a CDR2-alpha of MTFSENT (SEQ ID NO: 69) and a CDR3-alpha of SEQ ID NO: 22; and/or a beta alpha chain variable domain comprising a CDR1-beta of MNHNY (SEQ ID NO: 70), a CDR2-beta of SVGAGI (SEQ ID NO: 71) and a CDR3-beta of SEQ ID NO: 23.

The TCR may comprise an alpha chain variable domain comprising a CDR1-alpha of TSESDYY (SEQ ID NO: 72), a CDR2-alpha of QEAYKQQN (SEQ ID NO: 73) and a CDR3-alpha of SEQ ID NO: 24; and/or a beta alpha chain variable domain comprising a CDR1-beta of LNHDA (SEQ ID NO: 74), a CDR2-beta of SQIVND (SEQ ID NO: 75) and a CDR3-beta of SEQ ID NO: 25.

The TCR may comprise an alpha chain variable domain comprising a CDR1-alpha of TRDTTYY (SEQ ID NO: 76), a CDR2-alpha of RNSFDEQN (SEQ ID NO: 77) and a CDR3-alpha of SEQ ID NO: 26; and/or a beta alpha chain variable domain comprising a CDR1-beta of DFQATT (SEQ ID NO: 78), a CDR2-beta of SNEGSKA (SEQ ID NO: 79) and a CDR3-beta of SEQ ID NO: 27.

The TCR may comprise an alpha chain variable domain comprising a variable segment encoded by the TRAV38-1 gene, a Joining segment encoded by the TRAJ28 gene, a CDR1-alpha of SEQ ID NO: 28, a CDR2-alpha of SEQ ID NO: 29 and a CDR3-alpha of SEQ ID NO: 2; and/or a beta chain variable region comprising a variable segment encoded by the TRBV12-4 gene, a Joining segment encoded by the TRBJ1-2 gene, a CDR1-beta of SEQ ID NO: 30, a CDR2-beta of SEQ ID NO: 31 and a CDR3-beta of SEQ ID NO: 3.

The TCR may comprise an alpha chain variable domain comprising a variable segment encoded by the TRAV38-1 gene, a Joining segment encoded by the TRAJ43 gene, a CDR1-alpha of SEQ ID NO: 32, a CDR2-alpha of SEQ ID NO: 33 and a CDR3-alpha of SEQ ID NO: 4; and/or a beta chain variable region comprising a variable segment encoded by the TRBV5-6 gene, a Joining segment encoded by the TRBJ1-1 gene, a CDR1-beta of SEQ ID NO: 34, a CDR2-beta of SEQ ID NO: 35 and a CDR3-beta of SEQ ID NO: 5.

The TCR may comprise an alpha chain variable domain comprising a variable segment encoded by the TRAV38-1 gene, a Joining segment encoded by the TRAJ28 gene, a CDR1-alpha of SEQ ID NO: 36, a CDR2-alpha of SEQ ID NO: 37 and a CDR3-alpha of SEQ ID NO: 6; and/or a beta chain variable region comprising a variable segment encoded by the TRBV9 gene, a Joining segment encoded by the TRBJ1-6 gene, a CDR1-beta of SEQ ID NO: 38, a CDR2-beta of SEQ ID NO: 39 and a CDR3-beta of SEQ ID NO: 7.

The TCR may comprise an alpha chain variable domain comprising a variable segment encoded by the TRAV23DV6 gene, a Joining segment encoded by the TRAJ57 gene, a CDR1-alpha of SEQ ID NO: 40, a CDR2-alpha of SEQ ID NO: 41 and a CDR3-alpha of SEQ ID NO: 8; and/or a beta chain variable region comprising a variable segment encoded by the TRBV9 gene, a Joining segment encoded by the TRBJ1-6 gene, a CDR1-beta of SEQ ID NO: 42, a CDR2-beta of SEQ ID NO: 43 and a CDR3-beta of SEQ ID NO: 9.

The TCR may comprise an alpha chain variable domain comprising a variable segment encoded by the TRAV12-2 gene, a Joining segment encoded by the TRAJ48 gene, a CDR1-alpha of SEQ ID NO: 44, a CDR2-alpha of SEQ ID NO: 45 and a CDR3-alpha of SEQ ID NO: 10; and/or a beta chain variable region comprising a variable segment encoded by the TRBV28 gene, a Joining segment encoded by the TRBJ2-6 gene, a CDR1-beta of SEQ ID NO: 46, a CDR2-beta of SEQ ID NO: 47 and a CDR3-beta of SEQ ID NO: 11.

The TCR may comprise an alpha chain variable domain comprising a variable segment encoded by the TRAV38-1 gene, a Joining segment encoded by the TRAJ45 gene, a CDR1-alpha of SEQ ID NO: 48, a CDR2-alpha of SEQ ID NO: 49 and a CDR3-alpha of SEQ ID NO: 12; and/or a beta chain variable region comprising a variable segment encoded by the TRBV28 gene, a Joining segment encoded by the TRBJ2-3 gene, a CDR1-beta of SEQ ID NO: 50, a CDR2-beta of SEQ ID NO: 51 and a CDR3-beta of SEQ ID NO: 13.

The TCR may comprise an alpha chain variable domain comprising a variable segment encoded by the TRAV12-3 gene, a Joining segment encoded by the TRAJ29 gene, a CDR1-alpha of SEQ ID NO: 52, a CDR2-alpha of SEQ ID NO: 53 and a CDR3-alpha of SEQ ID NO: 14; and/or a beta chain variable region comprising a variable segment encoded by the TRBV2 gene, a Joining segment encoded by the TRBJ1-3 gene, a CDR1-beta of SEQ ID NO: 54, a CDR2-beta of SEQ ID NO: 55 and a CDR3-beta of SEQ ID NO: 15.

The TCR may comprise an alpha chain variable domain comprising a variable segment encoded by the TRAV13-1 gene, a Joining segment encoded by the TRAJ16 gene, a CDR1-alpha of SEQ ID NO: 56, a CDR2-alpha of SEQ ID NO: 57 and a CDR3-alpha of SEQ ID NO: 16; and/or a beta chain variable region comprising a variable segment encoded by the TRBV28 gene, a Joining segment encoded by the TRBJ2-2 gene, a CDR1-beta of SEQ ID NO: 58, a CDR2-beta of SEQ ID NO: 59 and a CDR3-beta of SEQ ID NO: 17.

The TCR may comprise an alpha chain variable domain comprising a variable segment encoded by the TRAV1-2 gene, a Joining segment encoded by the TRAJ34 gene, a CDR1-alpha of SEQ ID NO: 60, a CDR2-alpha of SEQ ID NO: 61 and a CDR3-alpha of SEQ ID NO: 18; and/or a beta chain variable region comprising a variable segment encoded by the TRBV7-9 gene, a Joining segment encoded by the TRBJ2-3 gene, a CDR1-beta of SEQ ID NO: 62, a CDR2-beta of SEQ ID NO: 63 and a CDR3-beta of SEQ ID NO: 19.

The TCR may comprise an alpha chain variable domain comprising a variable segment encoded by the TRAV29DV5 gene, a Joining segment encoded by the TRAJ22 gene, a CDR1-alpha of SEQ ID NO: 64, a CDR2-alpha of SEQ ID NO: 65 and a CDR3-alpha of SEQ ID NO: 20; and/or a beta chain variable region comprising a variable segment encoded by the TRBVS-1 gene, a Joining segment encoded by the TRBJ2-6 gene, a CDR1-beta of SEQ ID NO: 66, a CDR2-beta of SEQ ID NO: 67 and a CDR3-beta of SEQ ID NO: 21.

The TCR may comprise an alpha chain variable domain comprising a variable segment encoded by the TRAV10 gene, a Joining segment encoded by the TRAJ17 gene, a CDR1-alpha of SEQ ID NO: 68, a CDR2-alpha of SEQ ID NO: 69 and a CDR3-alpha of SEQ ID NO: 22; and/or a beta chain variable region comprising a variable segment encoded by the TRBV6-6 gene, a Joining segment encoded by the TRBJ2-3 gene, a CDR1-beta of SEQ ID NO: 70, a CDR2-beta of SEQ ID NO: 71 and a CDR3-beta of SEQ ID NO: 23.

The TCR may comprise an alpha chain variable domain comprising a variable segment encoded by the TRAV38-2DV8 gene, a Joining segment encoded by the TRAJ43 gene, a CDR1-alpha of SEQ ID NO: 72, a CDR2-alpha of SEQ ID NO: 73 and a CDR3-alpha of SEQ ID NO: 24; and/or a beta chain variable region comprising a variable segment encoded by the TRBV19 gene, a Joining segment encoded by the TRBJ1-5 gene, a CDR1-beta of SEQ ID NO: 74, a CDR2-beta of SEQ ID NO: 75 and a CDR3-beta of SEQ ID NO: 25.

The TCR may comprise an alpha chain variable domain comprising a variable segment encoded by the TRAV19 gene, a Joining segment encoded by the TRAJ40 gene, a CDR1-alpha of SEQ ID NO: 76, a CDR2-alpha of SEQ ID NO: 77 and a CDR3-alpha of SEQ ID NO: 26; and/or a beta chain variable region comprising a variable segment encoded by the TRBV20 gene, a Joining segment encoded by the TRBJ2-7 gene, a CDR1-beta of SEQ ID NO: 78, a CDR2-beta of SEQ ID NO: 79 and a CDR3-beta of SEQ ID NO: 27.

The TCR may comprise an alpha chain variable domain comprising a variable segment encoded by the TRAV41 gene, a Joining segment encoded by the TRAJ57 gene, a CDR3-alpha of SEQ ID NO: 111; and/or a beta chain variable region comprising a variable segment encoded by the TRBV9 gene, a Joining segment encoded by the TRBJ2-3 gene, and a CDR3-beta of SEQ ID NO: 112.

The TCR may comprise an alpha chain variable domain comprising a variable segment encoded by the TRAV1-2 gene, a Joining segment encoded by the TRAJ31 gene, a CDR3-alpha of SEQ ID NO: 113; and/or a beta chain variable region comprising a variable segment encoded by the TRBV20-1 gene, a Joining segment encoded by the TRBJ1-2 gene, and a CDR3-beta of SEQ ID NO: 114.

The TCR may comprise an alpha chain variable domain comprising a variable segment encoded by the TRAV35 gene, a Joining segment encoded by the TRAJ45 gene, a CDR3-alpha of SEQ ID NO: 115; and/or a beta chain variable region comprising a variable segment encoded by the TRBV5-6 gene, a Joining segment encoded by the TRBJ2-5 gene, and a CDR3-beta of SEQ ID NO: 116.

The TCR may comprise an alpha chain variable domain comprising a variable segment encoded by the TRAV19 gene, a Joining segment encoded by the TRAJ32 gene, a CDR3-alpha of SEQ ID NO: 117; and/or a beta chain variable region comprising a variable segment encoded by the TRBV15 gene, a Joining segment encoded by the TRBJ1-1 gene, and a CDR3-beta of SEQ ID NO: 118.

The TCR may comprise an alpha chain variable domain comprising a variable segment encoded by the TRAV41 gene, a Joining segment encoded by the TRAJ45 gene, a CDR3-alpha of SEQ ID NO: 119; and/or a beta chain variable region comprising a variable segment encoded by the TRBV29-1 gene, a Joining segment encoded by the TRBJ1-1 gene, and a CDR3-beta of SEQ ID NO: 120.

The TCR may comprise an alpha chain variable domain comprising a variable segment encoded by the TRAV19 gene, a Joining segment encoded by the TRAJ28 gene, a CDR3-alpha of SEQ ID NO: 121; and/or a beta chain variable region comprising a variable segment encoded by the TRBV19 gene, a Joining segment encoded by the TRBJ1-5 gene, and a CDR3-beta of SEQ ID NO: 122.

The TCR may comprise an alpha chain variable domain comprising a variable segment encoded by the TRAV41 gene, a Joining segment encoded by the TRAJ48 gene, a CDR3-alpha of SEQ ID NO: 123; and/or a beta chain variable region comprising a variable segment encoded by the TRBV9 gene, a Joining segment encoded by the TRBJ2-2 gene, and a CDR3-beta of SEQ ID NO: 124.

The TCR may comprise an alpha chain variable domain comprising a variable segment encoded by the TRAV13-2 gene, a Joining segment encoded by the TRAJ32 gene, a CDR3-alpha of SEQ ID NO: 125; and/or a beta chain variable region comprising a variable segment encoded by the TRBV2 gene, a Joining segment encoded by the TRBJ2-7 gene, and a CDR3-beta of SEQ ID NO: 126.

The TCR may comprise an alpha chain variable domain comprising a variable segment encoded by the TRAV25 gene, a Joining segment encoded by the TRAJ54 gene, a CDR3-alpha of SEQ ID NO: 127; and/or a beta chain variable region comprising a variable segment encoded by the TRBV27 gene, a Joining segment encoded by the TRBJ2-7 gene, and a CDR3-beta of SEQ ID NO: 128.

The TCR may comprise an alpha chain variable domain comprising a variable segment encoded by the TRAV3 gene, a Joining segment encoded by the TRAJ21 gene, a CDR3-alpha of SEQ ID NO: 129; and/or a beta chain variable region comprising a variable segment encoded by the TRBV28 gene, a Joining segment encoded by the TRBJ1-4 gene, and a CDR3-beta of SEQ ID NO: 130.

The TCR may comprise an alpha chain variable domain comprising a variable segment encoded by the TRAV14DV4 gene, a Joining segment encoded by the TRAJ28 gene, a CDR3-alpha of SEQ ID NO: 131; and/or a beta chain variable region comprising a variable segment encoded by the TRBV9 gene, a Joining segment encoded by the TRBJ1-1 gene, and a CDR3-beta of SEQ ID NO: 132.

The TCR may comprise an alpha chain variable domain comprising a variable segment encoded by the TRAV12-2 gene, a Joining segment encoded by the TRAJ10 gene, a CDR3-alpha of SEQ ID NO: 133; and/or a beta chain variable region comprising a variable segment encoded by the TRBV2 gene, a Joining segment encoded by the TRBJ2-7 gene, and a CDR3-beta of SEQ ID NO: 134.

The TCR may comprise an alpha chain variable domain comprising a variable segment encoded by the TRAV39 gene, a Joining segment encoded by the TRAJ31 gene, a CDR3-alpha of SEQ ID NO: 135; and/or a beta chain variable region comprising a variable segment encoded by the TRBV10-3 gene, a Joining segment encoded by the TRBJ1-2 gene, and a CDR3-beta of SEQ ID NO: 136.

The TCR may comprise an alpha chain variable domain comprising a variable segment encoded by the TRAV29DV5 gene, a Joining segment encoded by the TRAJ22 gene, a CDR3-alpha of SEQ ID NO: 137; and/or a beta chain variable region comprising a variable segment encoded by the TRBV9 gene, a Joining segment encoded by the TRBJ2-3 gene, and a CDR3-beta of SEQ ID NO: 138.

The TCR may comprise an alpha chain variable domain comprising a variable segment encoded by the TRAV19 gene, a Joining segment encoded by the TRAJ11 gene, a CDR3-alpha of SEQ ID NO: 139; and/or a beta chain variable region comprising a variable segment encoded by the TRBV6-6 gene, a Joining segment encoded by the TRBJ2-7 gene, and a CDR3-beta of SEQ ID NO: 140.

The TCR may comprise an alpha chain variable domain comprising a variable segment encoded by the TRAV21 gene, a Joining segment encoded by the TRAJ37 gene, a CDR3-alpha of SEQ ID NO: 141; and/or a beta chain variable region comprising a variable segment encoded by the TRBV29-1 gene, a Joining segment encoded by the TRBJ2-7 gene, and a CDR3-beta of SEQ ID NO: 142.

The TCR may comprise an alpha chain variable domain comprising a variable segment encoded by the TRAV23DV6 gene, a Joining segment encoded by the TRAJ30 gene, a CDR3-alpha of SEQ ID NO: 143; and/or a beta chain variable region comprising a variable segment encoded by the TRBV27 gene, a Joining segment encoded by the TRBJ1-1 gene, and a CDR3-beta of SEQ ID NO: 144.

The TCR may comprise an alpha chain variable domain comprising a variable segment encoded by the TRAV21 gene, a Joining segment encoded by the TRAJ28 gene, a CDR3-alpha of SEQ ID NO: 145; and/or a beta chain variable region comprising a variable segment encoded by the TRBV27 gene, a Joining segment encoded by the TRBJ2-3 gene, and a CDR3-beta of SEQ ID NO: 146.

The TCR may comprise an alpha chain variable domain comprising a variable segment encoded by the TRAV21 gene, a Joining segment encoded by the TRAJ57 gene, a CDR3-alpha of SEQ ID NO: 147; and/or a beta chain variable region comprising a variable segment encoded by the TRBV28 gene, a Joining segment encoded by the TRBJ1-2 gene, and a CDR3-beta of SEQ ID NO: 148.

The TCR may comprise an alpha chain variable domain of a sequence of MTRVSLLWAVVVSTCLESGMAQTVTQSQPEMSVQEAETVTLSCTYDTSENNYY LFWYKQPPSRQMILVIRQEAYKQQNATENRFSVNFQKAAKSFSLKISDSQLGDTA MYFCAFMKLHSGAGSYQLTFGKGTKLSVIP (SEQ ID NO: 80) or a sequence with at least 90% identity, such as 90%, 95%, 96%, 97%, 98%, 99% of 100% identity to SEQ ID NO: 80, and a beta chain variable domain of a sequence of MGSWTLCCVSLCILVAKHTDAGVIQSPRHEVTEMGQEVTLRCKPISGHDYLFWY RQTMMRGLELLIYFNNNVPIDDSGMPEDRFSAKMPNASFSTLKIQPSEPRDSAVY FCASSLWAVGYGYTFGSGTRLTVV (SEQ ID NO: 81) or a sequence with at least 90% identity, such as 90%, 95%, 96%, 97%, 98%, 99% of 100% identity to SEQ ID NO: 81.

The TCR may comprise an alpha chain variable domain of a sequence of MTRVSLLWAVVVSTCLESGMAQTVTQSQPEMSVQEAETVTLSCTYDTSENNYY LFWYKQPPSRQMILVIRQEAYKQQNATENRFSVNFQKAAKSFSLKISDSQLGDTA MYFCAFDNNNDMRFGAGTRLTVKP (SEQ ID NO: 82) or a sequence with at least 90% identity, such as 90%, 95%, 96%, 97%, 98%, 99% of 100% identity to SEQ ID NO: 82, and a beta chain variable domain of a sequence of MGPGLLCWALLCLLGAGLVDAGVTQSPTHLIKTRGQQVTLRCSPKSGHDTVSW YQQALGQGPQFIFQYYEEEERQRGNFPDRFSGHQFPNYSSELNVNALLLGDSALY LCASSLVGAITEAFFGQGTRLTVV (SEQ ID NO: 83) or a sequence with at least 90% identity, such as 90%, 95%, 96%, 97%, 98%, 99% of 100% identity to SEQ ID NO: 83.

The TCR may comprise an alpha chain variable domain of a sequence of MTRVSLLWAVVVSTCLESGMAQTVTQSQPEMSVQEAETVTLSCTYDTSENNYY LFWYKQPPSRQMILVIRQEAYKQQNATENRFSVNFQKAAKSFSLKISDSQLGDTA MYFCAFVDGAGSYQLTFGKGTKLSVIP (SEQ ID NO: 84) or a sequence with at least 90% identity, such as 90%, 95%, 96%, 97%, 98%, 99% of 100% identity to SEQ ID NO: 84, and a beta chain variable domain of a sequence of MGFRLLCCVAFCLLGAGPVDSGVTQTPKHLITATGQRVTLRCSPRSGDLSVYWY QQSLDQGLQFLIQYYNGEERAKGNILERFSAQQFPDLHSELNLSSLELGDSALYFC ASSVGNSNSPLHFGNGTRLTVT (SEQ ID NO: 85) or a sequence with at least 90% identity, such as 90%, 95%, 96%, 97%, 98%, 99% of 100% identity to SEQ ID NO: 85.

The TCR may comprise an alpha chain variable domain of a sequence of MDKILGASFLVLWLQLCWVSGQQKEKSDQQQVKQSPQSLIVQKGGISIINCAYEN TAFDYFPWYQQFPGKGPALLIAIRPDVSEKKEGRFTISFNKSAKQFSLHIMDSQPG DSATYFCAASGLFIQGGSEKLVFGKGMKLTVNP (SEQ ID NO: 86) or a sequence with at least 90% identity, such as 90%, 95%, 96%, 97%, 98%, 99% of 100% identity to SEQ ID NO: 86, and a beta chain variable domain of a sequence of MGFRLLCCVAFCLLGAGPVDSGVTQTPKHLITATGQRVTLRCSPRSGDLSVYWY QQSLDQGLQFLIQYYNGEERAKGNILERFSAQQFPDLHSELNLSSLELGDSALYFC ASSVGNSNSPLHFGNGTRLTVT (SEQ ID NO: 87) or a sequence with at least 90% identity, such as 90%, 95%, 96%, 97%, 98%, 99% of 100% identity to SEQ ID NO: 87.

The TCR may comprise an alpha chain variable domain of a sequence of MKSLRVLLVILWLQLSWVWSQQKEVEQNSGPLSVPEGAIASLNCTYSDRGSQSFF WYRQYSGKSPELIMFIYSNGDKEDGRFTAQLNKASQYVSLLIRDSQPSDSATYLC AVYGSGKLTFGTGTRLTIIP (SEQ ID NO: 88) or a sequence with at least 90% identity, such as 90%, 95%, 96%, 97%, 98%, 99% of 100% identity to SEQ ID NO: 88, and a beta chain variable domain of a sequence of MGIRLLCRVAFCFLAVGLVDVKVTQSSRYLVKRTGEKVFLECVQDMDHENMFW YRQDPGLGLRLIYFSYDVKMKEKGDIPEGYSVSREKKERFSLILESASTNQTSMY LCASSFGPSSGANVLTFGAGSRLTVL (SEQ ID NO: 89) or a sequence with at least 90% identity, such as 90%, 95%, 96%, 97%, 98%, 99% of 100% identity to SEQ ID NO: 89.

The TCR may comprise an alpha chain variable domain of a sequence of MTRVSLLWAVVVSTCLESGMAQTVTQSQPEMSVQEAETVTLSCTYDTSENNYY LFWYKQPPSRQMILVIRQEAYKQQNATENRFSVNFQKAAKSFSLKISDSQLGDTA MYFCAFTLYSGGGADGLTFGKGTHLIIQP (SEQ ID NO: 90) or a sequence with at least 90% identity, such as 90%, 95%, 96%, 97%, 98%, 99% of 100% identity to SEQ ID NO: 90, and a beta chain variable domain of a sequence of MGIRLLCRVAFCFLAVGLVDVKVTQSSRYLVKRTGEKVFLECVQDMDHENMFW YRQDPGLGLRLIYFSYDVKMKEKGDIPEGYSVSREKKERFSLILESASTNQTSMY LCASSLPTSLSTDTQYFGPGTRLTVL (SEQ ID NO: 91) or a sequence with at least 90% identity, such as 90%, 95%, 96%, 97%, 98%, 99% of 100% identity to SEQ ID NO: 91.

The TCR may comprise an alpha chain variable domain of a sequence of MMKSLRVLLVILWLQLSWVWSQQKEVEQDPGPLSVPEGAIVSLNCTYSNSAFQY FMWYRQYSRKGPELLMYTYSSGNKEDGRFTAQVDKSSKYISLFIRDSQPSDSATY LCAMSWNSGNTPLVFGKGTRLSVIA (SEQ ID NO: 92) or a sequence with at least 90% identity, such as 90%, 95%, 96%, 97%, 98%, 99% of 100% identity to SEQ ID NO: 92, and a beta chain variable domain of a sequence of MDTWLVCWAIFSLLKAGLTEPEVTQTPSHQVTQMGQEVILRCVPISNHLYFYWY RQILGQKVEFLVSFYNNEISEKSEIFDDQFSVERPDGSNFTLKIRSTKLEDSAMYFC ASSVTGVRNTIYFGEGSWLTVV (SEQ ID NO: 93) or a sequence with at least 90% identity, such as 90%, 95%, 96%, 97%, 98%, 99% of 100% identity to SEQ ID NO: 93.

The TCR may comprise an alpha chain variable domain of a sequence of MTSIRAVFIFLWLQLDLVNGENVEQHPSTLSVQEGDSAVIKCTYSDSASNYFPWY KQELGKRPQLIIDIRSNVGEKKDQRIAVTLNKTAKHFSLHITETQPEDSAVYFCAA YGQKLLFARGTMLKVDL (SEQ ID NO: 94) or a sequence with at least 90% identity, such as 90%, 95%, 96%, 97%, 98%, 99% of 100% identity to SEQ ID NO: 94, and a beta chain variable domain of a sequence of MGIRLLCRVAFCFLAVGLVDVKVTQSSRYLVKRTGEKVFLECVQDMDHENMFW YRQDPGLGLRLIYFSYDVKMKEKGDIPEGYSVSREKKERFSLILESASTNQTSMY LCASSLLEPDLNTGELFFGEGSRLTVL (SEQ ID NO: 95) or a sequence with at least 90% identity, such as 90%, 95%, 96%, 97%, 98%, 99% of 100% identity to SEQ ID NO: 95.

The TCR may comprise an alpha chain variable domain of a sequence of MWGVFLLYVSMKMGGTTGQNIDQPTEMTATEGAIVQINCTYQTSGFNGLFWYQ QHAGEAPTFLSYNVLDGLEEKGRFSSFLSRSKGYSYLLLKELQMKDSASYLCAV NTDKLIFGTGTRLQVFP (SEQ ID NO: 96) or a sequence with at least 90% identity, such as 90%, 95%, 96%, 97%, 98%, 99% of 100% identity to SEQ ID NO: 96, and a beta chain variable domain of a sequence of MGTSLLCWMALCLLGADHADTGVSQDPRHKITKRGQNVTFRCDPISEHNRLYW YRQTLGQGPEFLTYFQNEAQLEKSRLLSDRFSAERPKGSFSTLEIQRTEQGDSAM YLCASSNPGNSDFGPGTRLTVL (SEQ ID NO: 97) or a sequence with at least 90% identity, such as 90%, 95%, 96%, 97%, 98%, 99% of 100% identity to SEQ ID NO: 97.

The TCR may comprise an alpha chain variable domain of a sequence of MAMLLGASVLILWLQPDWVNSQQKNDDQQVKQNSPSLSVQEGRISILNCDYTNS MFDYFLWYKKYPAEGPTFLISISSIKDKNEDGRFTVFLNKSAKHLSLHIVPSQPGD SAVYFCAAVSTGSARQLTFGSGTQLTVLP (SEQ ID NO: 98) or a sequence with at least 90% identity, such as 90%, 95%, 96%, 97%, 98%, 99% of 100% identity to SEQ ID NO: 98, and a beta chain variable domain of a sequence of MGSRLLCWVLLCLLGAGPVKAGVTQTPRYLIKTRGQQVTLSCSPISGHRSVSWY QQTPGQGLQFLFEYFSETQRNKGNFPGRFSGRQFSNSRSEMNVSTLELGDSALYL CASSLAKGANVLTFGAGSRLTVL (SEQ ID NO: 99) or a sequence with at least 90% identity, such as 90%, 95%, 96%, 97%, 98%, 99% of 100% identity to SEQ ID NO: 99.

The TCR may comprise an alpha chain variable domain of a sequence of MKKHLTTFLVILWLYFYRGNGKNQVEQSPQSLIILEGKNCTLQCNYTVSPFSNLR WYKQDTGRGPVSLTIMTFSENTKSNGRYTATLDADTKQSSLHITASQLSDSASYI CVVSAWDPAAGNKLTFGGGTRVLVKP (SEQ ID NO: 100) or a sequence with at least 90% identity, such as 90%, 95%, 96%, 97%, 98%, 99% of 100% identity to SEQ ID NO: 100, and a beta chain variable domain of a sequence of MSISLLCCAAFPLLWAGPVNAGVTQTPKFRILKIGQSMTLQCTQDMNHNYMYW YRQDPGMGLKLIYYSVGAGITDKGEVPNGYNVSRSTTEDFPLRLELAAPSQTSVY FCASSPGGQGLDTQYFGPGTRLTVL (SEQ ID NO: 101) or a sequence with at least 90% identity, such as 90%, 95%, 96%, 97%, 98%, 99% of 100% identity to SEQ ID NO: 101.

The TCR may comprise an alpha chain variable domain of a sequence of MACPGFLWALVISTCLEFSMAQTVTQSQPEMSVQEAETVTLSCTYDTSESDYYLF WYKQPPSRQMILVIRQEAYKQQNATENRFSVNFQKAAKSFSLKISDSQLGDAAM YFCAYNRNDMRFGAGTRLTVKP (SEQ ID NO: 102) or a sequence with at least 90% identity, such as 90%, 95%, 96%, 97%, 98%, 99% of 100% identity to SEQ ID NO: 102, and a beta chain variable domain of a sequence of MSNQVLCCVVLCFLGANTVDGGITQSPKYLFRKEGQNVTLSCEQNLNHDAMYW YRQDPGQGLRLIYYSQIVNDFQKGDIAEGYSVSREKKESFPLTVTSAQKNPTAFY LCASSTDRDNQPQHFGDGTRLSIL (SEQ ID NO: 103) or a sequence with at least 90% identity, such as 90%, 95%, 96%, 97%, 98%, 99% of 100% identity to SEQ ID NO: 103.

The TCR may comprise an alpha chain variable domain of a sequence of MLTASLLRAVIASICVVSSMAQKVTQAQTEISVVEKEDVTLDCVYETRDTTYYLF WYKQPPSGELVFLIRRNSFDEQNEISGRYSWNFQKSTSSFNFTITASQVVDSAVYF CALSEALTSGTYKYIFGTGTRLKVLA (SEQ ID NO: 104) or a sequence with at least 90% identity, such as 90%, 95%, 96%, 97%, 98%, 99% of 100% identity to SEQ ID NO: 104 and a beta chain variable domain of a sequence of MLLLLLLLGPGISLLLPGSLAGSGLGAVVSQHPSWVICKSGTSVKIECRSLDFQAT TMFWYRQFPKQSLMLMATSNEGSKATYEQGVEKDKFLINHASLTLSTLTVTSAH PEDSSFYICSASVGKSSYEQYVGPGTRLTVT (SEQ ID NO: 105) or a sequence with at least 90% identity, such as 90%, 95%, 96%, 97%, 98%, 99% of 100% identity to SEQ ID NO: 105.

The TCR may comprise an alpha chain TRAC constant domain sequence and/or a beta chain TRBC1 or TRBC2 constant domain sequence. The constant domain may be from another species, such as mouse TRAC and or TRBC, or from a human. A mouse constant domain may allow for monitoring of the the survival of T cells comprising such a TCR when injected in vivo, for example. The hybrid TCRs will also only pair together, which avoids hybrid TCR alpha or beta chain molecules pairing with the host cell TCRs alpha or beta chain molecules.

The TCR may be a recombinant TCR. The TCR may be a soluble TCR.

In another aspect, there is provided a recombinant nucleic acid encoding one or more, TCR disclosed herein, such as one, two, three, four, five, six, seven, eight, nine, ten, eleven, twelve, thirteen or more TCRs disclosed herein.

The recombinant nucleic acid may be a DNA or RNA molecule. The DNA may be a cDNA. The nucleic acid may be non-naturally occurring and/or purified and/or engineered

In another aspect, there is provided a vector comprising a nucleic acid disclosed herein. The vector may be a TCR expression vector. The vector may be a plasmid. The vector may be a viral vector, such as a retroviral vector, lentiviral vector or adenoviral vector. The vector may comprise nucleic acid disclosed herein, encoding in a single open reading frame, or two distinct open reading frames, the alpha chain and the beta chain respectively.

In another aspect, there is provided a cell or population of cells comprising and/or encoding one or more TCR, nucleic acid or vector disclosed herein. The cell or population of cells may harbour a first expression vector which comprises nucleic acid encoding the alpha chain of a TCR disclosed herein, and a second expression vector comprising nucleic acid encoding the beta chain of a TCR disclosed herein.

The cell may be a T-cell. The T-cell may be a CD4+ T cell. The T-cell may be a CD8+ T cell. The cell or population of cells may include a non-naturally occurring and/or purified and/or or engineered cell, especially a T-cell such as a CD8+ T-cell, expressing/presenting a TCR disclosed herein. There are a number of methods suitable for the transfection of T cells with nucleic acid (such as DNA, cDNA or RNA) encoding the TCRs disclosed herein (see for example Robbins et al., (2008) J Immunol. 180: 6 116-61 3 1). T-cells expressing the TCRs disclosed herein will be suitable for use in adoptive therapy-based treatment of or prevention of HIV-1 and/or AIDS. As will be known to those skilled in the art, there are a number of suitable methods by which adoptive therapy can be carried out (see for example Rosenberg et al, (2008) Nat Rev Cancer 8(4): 299-308).

The cells or population of cells may be isolated and/or recombinant and/or non-naturally occurring and/or engineered.

In another aspect, there is provided a pharmaceutical composition comprising one or more TCR, nucleic acid, vector, cell or population of cells disclosed herein.

In another aspect, there is provided a TCR, nucleic acid, vector, cell or population of cells, or pharmaceutical composition disclosed herein for use in treating or preventing virus infection in a subject. The virus may be HIV. The HIV may be HIV-1 or HIV-2. A TCR, nucleic acid, vector, cell or population of cells, or pharmaceutical composition disclosed herein may be for use in treating or preventing HIV-1 infection and/or AIDS.

In another aspect, there is provided a method of treating or preventing virus infection in a subject, comprising administering to the subject one or more TCR, nucleic acid, vector, cell or population of cells, or composition disclosed herein. The virus may be HIV. The HIV may be HIV-1 or HIV-2. The method may be to treat or prevent HIV-1 infection and/or AIDS.

In another aspect, there the use of or more TCR, nucleic acid, vector, cell or population of cells, or pharmaceutical composition disclosed herein in the manufacture of a medicament for treating or preventing virus infection n a subject. The virus may be HIV. The HIV may be HIV-1 or HIV-2. The use of a TCR, nucleic acid, vector, cell or population of cells, or pharmaceutical composition disclosed herein in the manufacture of a medicament may be for treating or preventing HIV-1 and/or AIDS may be for use in treating or preventing HIV-1 infection and/or AIDS.

In another aspect, there is provided a combination therapeutic for use in treating or preventing of virus infection in a subject, comprising:
a) the TCR, nucleic acid, vector, cell or population of cells, or pharmaceutical composition disclosed herein; and
b) one or more further therapeutic agent.

The virus may be HIV. The HIV may be HIV-1 or HIV-2. The combination therapeutic may be for use in treating or preventing HIV-1 infection and/or AIDS.

The one or more further therapeutic agent may be an agent known for the treatment and/or prevention of HIV-1 infection and/or AIDS. The one or more further therapeutic agent may be a cytotoxic agent. The one or more further reagent could be an agent that activates latent HIV-1 in viral reservoirs. The one or more further reagent could be an immune checkpoint inhibitor. The one or more further reagent could be an antibody or receptor mimic that neutralizes or inactivates HIV-1. The one or more further therapeutic agent may be an immunomodulatory agent, such as IL-2, IFN-gamma, an anti-CD3 antibody.

TCRs may be used, as soluble targeting agents for the purpose of delivering cytotoxic or immune effector agents to HIV-1 infected cells (Lissin, et al., (2013). “High-Affinity Monocloncal T-cell receptor (mTCR) Fusions. Fusion Protein Technologies for Biophamaceuticals: Applications and Challenges”. S. R. Schmidt, Wiley; Boulter, et al',(2003), Protein Eng 16(9): 707-71 1; Liddy, et al.,(2012), Nat Med 8 : 980-987), or alternatively they may be used to engineer T cells for adoptive therapy (June, et al., (2014), Cancer Immunol Immunother 63(9): 969-975).

In the cell or population of cells of any aspect, the endogenous TCR may be removed or inactivated.

In any aspect where virus infection, such as HIV-1 infection and/or AIDS, is to be treated or prevented, the cell or population of cells may be derived from the subject or from an allogeneic donor who is not infected with the virus, such as HIV-1.

The subject may be a mammal, preferably a human. The subject may already be infected with the virus to be treated or prevented, or may be at risk of being infected with the virus to be treated or prevented. The subject may already be infected with HIV-1 or may be at risk of being infected with HIV-1.

### DETAILED DESCRIPTION

The invention is in part based on the finding that human CD8+ T cells can be generated to respond to an HIV-1 Gag peptide or Rev peptide presented by HLA-E, and that HIV-1 infected CD4 T+ cells present this epitope and are suppressed by HLA-E restricted T cells. Specifically, the inventors stimulated primary T cell responses to the HIV-1 RL9 or IL9 (ILVESPAVL) peptide in vitro, using peripheral blood mononuclear cells (PBMCs) from HIV uninfected donors. CD8+ T cell clones that were HLA-E restricted and RL9 or IL9 specific were grown; these cells were polyfunctional and suppressed HIV replication in vitro. Importantly, the inventors demonstrate for the first time that HLA-E restricted HIV specific T cells are capable of recognising HIV infected cells can be primed in humans.

Traditionally, the non-classical human HLA class I molecule HLA-E is associated with the immune monitoring by NK cells. This process involves the presentation of a specific nonamer peptide derived from residues 3-11 of the classical HLA class Ia signal sequences, typically VMAPRTLVL (VL9), by HLA-E at the cell surface. This complex is recognised by the inhibitory receptor NKG2A-CD94 and its activating counterpart NKG2C-CD94, which are expressed on natural killer (NK) cells and a subset of CD8+ T cells. The balance between the inhibitory and activating signals regulates NK cell activity and their ability to kill cells that do not express classical HLA class I molecules. Binding of the homologous signal peptide and recognition by the NKG2-CD94 receptors on NK cells is conserved for the closely related non-classical MHC molecules, H-2Qa-1 in mice, and Mamu-E in rhesus macaques. Furthermore, HLA-E and Mamu-E are specifically targeted by human and rhesus CMVs, thus, regulation of NK cell activity appears to be the principle function of the MHC-E family of molecules across species.

Further, it is very uncommon that immune responses to known infections are driven by HLA-E restricted CD8+ T cells; only a few are currently known. These include HLA-E restricted T cells specific for mycobacterial peptide antigens are found in most adult humans and more than 70 epitopes have been identified; The HLA-E restricted response is a major component of the CD8+ T cell response to this microbe. Secondly, Mamu-E restricted T cell responses were identified in rhesus macaques vaccinated with rhesus cytomegalovirus strain 68-1, which was recombinant for all SIV genes except Vif (RhCMV 68-1-SIV) (S. G. Hansen et al., Broadly targeted CD8(+) T cell responses restricted by majorhistocompatibility complex E. Science 351, 714-720 (2016)). Atypical MHC class II restricted CD8+ T cell responses were also detected in vaccinated macaques, but classical Class Ia restricted CD8 T cell responses specific for SIV or CMV were absent (S. G. Hansen et al., Cytomegalovirus vectors violate CD8+ T cell epitope recognition paradigms. Science 340, 1237874 (2013)). When these animals were challenged with pathogenic SIVmac239 one year after immunization and infection with the persisting RhCMV68-1 vaccine, all animals became SIV infected, but remarkably over half cleared SIV completely within weeks. Up to now, HIV specific HLA-E restricted T cells have not been described in natural HIV-1 infection.

As there are only two HLA-E alleles present in humans, both with identical peptide binding grooves, the invention provides a further benefit that all humans could respond to the same peptide epitopes, thus offering the potential to target universal peptide epitopes via HLA-E so that the therapeutic could treat all patients.

Additionally, as CD8+ T-cells are highly affected by the persistent immune activation and exhaustion state driven by the increased antigenic and inflammatory burden during HIV infection, the invention provides an opportunity to produce HLA-E restricted RL9- or IL9-specific TCRs and T-cells expressing such TCRs from third party blood donors on an industrial scale, providing an “off the shelf” opportunity to negate the effects of T-cell exhaustion in the host.

Within the scope of the invention are phenotypically silent variants of any TCR disclosed herein. As used herein the term “phenotypically silent variants” is understood to refer to a TCR which incorporates one or more further amino acid changes, in which a TCR has a similar phenotype to the corresponding TCR without said change(s). As is known to those skilled in the art, it may be possible to produce TCRs that incorporate changes in the constant and/or variable domains thereof compared to those detailed above without altering the affinity for the interaction with the SEQ ID NO:1: HLA-E complex. In particular, such silent mutations may be incorporated within parts of the sequence that are known not to be directly involved in antigen binding (e.g. outside the CDRs). Such trivial variants are included in the scope of this invention. Those TCRs in which one or more conservative substitutions have been made also form part of this invention.

Mutagenesis can be carried out using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) and restriction enzyme-based cloning, see Sambrook & Russell, (2001) Molecular Cloning—A Laboratory Manual (3rd Ed.) CSHL Press. Further information on ligation independent cloning (LIC) procedures can be found in Rashtchian, (1995) Curr Opin Biotechnol 6(1): 30-6.

The TCR may be an alpha-beta heterodimer, or may be in single chain format. Single chain formats include αβ TCR polypeptides of the Va-L-Vβ, Vβ-L-Va, Va-Ca-L-Vβ, Va-L-Vβ-Cβ or Va-Ca-L-Vβ-Cβ or Vβ-Cβ-L-Va-Ca types, wherein Va and Vβ are TCR a and β variable regions respectively, C and β are TCR a and β constant regions respectively, and L is a linker sequence. For use as a targeting agent for delivering therapeutic agents to the antigen presenting cell the TCR may be in soluble form (i.e. having no transmembrane or cytoplasmic domains). For stability, soluble β heterodimeric TCRs preferably have an introduced disulphide bond between residues of the respective constant domains, as described, for example, in WO 03/020763. One or both of the constant domains present in an αβ heterodimer of a TCR disclosed herein may be truncated at the C terminus or C termini, for example by up to 15, or up to 10 or up to 8 or fewer amino acids. For use in adoptive therapy, an αβ heterodimeric TCR may, for example, be transfected as full-length chains having both cytoplasmic and transmembrane domains. TCRs for use in adoptive therapy may contain a disulphide bond corresponding to that found in nature between the respective alpha and beta constant domains, additionally or alternatively a non-native disulphide bond may be present. As will be obvious to those skilled in the art, it may be possible to truncate the sequences provided at the C-terminus and/or N-terminus thereof, by 1, 2 , 3 , 4 , 5 or more residues, without substantially affecting the binding characteristics of the TCR. All such trivial variants are encompassed by the present invention.

A TCR disclosed herein may also be part of a multivalent complex of the TCR. Such multivalent TCR complexes may be particularly useful for tracking or targeting cells presenting particular antigens in vitro or in vivo, and are also useful as intermediates for the production of further multivalent TCR complexes having such uses.

Any sequence referred to herein, such as any of SEQ ID NOs: 1-109 may also encompass a sequence which has at least 90% identity, such as 90%, 95%, 96%, 97%, 98%, 99% of 100% identity to any of SEQ ID NOs: 1-109.

“Identity” as known in the art is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic Acids Research, 12, 387 (1984), BLASTP, BLASTN, and FASTA (Atschul et al., J. Molec. Biol. 215, 403 (1990)).

A program such as the CLUSTAL program can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of identity analysis are suitable.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad . Sci. USA 87:2264-2268, modified as in Karlin and Altschul (1993) Proc. Natl. Acad. Sci. USA 90:5873- 5877. The NBLAST and XBLAST programs of Altschul, et al. (1990) J. Mol. Biol. 2 15:403-41 0 have incorporated such an algorithm. BLAST nucleotide searches can be performed with the NBLAST program, score=100, wordlength=12 to obtain nucleotide sequences homologous to nucleic acid molecules. BLAST protein searches can be performed with the XBLAST program, score=50, wordlength=3 to obtain amino acid sequences homologous to protein molecules for use in the invention. To obtain gapped alignments for comparison purposes, Gapped BLAST can be utilised as described in Altschul et al. (1997) Nucleic Acids Res. 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., XBLAST and NBLAST) can be used. See http://www.ncbi.nlm.nih.gov. Another example of a mathematical algorithm utilised for the comparison of sequences is the algorithm of Myers and Miller, CABIOS (1989). The ALIGN program (version 2.0) which is part of the CGC sequence alignment software package has incorporated such an algorithm. Other algorithms for sequence analysis known in the art include ADVANCE and ADAM as described in Torellis and Robotti (1994) Comput. Appl. Biosci., 10 :3-5; and FASTA described in Pearson and Lipman (1988) Proc. Natl. Acad. Sci. 85:2444-8. Within FASTA, ktup is a control option that sets the sensitivity and speed of the search.

TCR specificity can be measured in vitro, for example, in cellular assays known to the skilled person. To test specificity the TCRs may be in soluble form and/or may be expressed on the surface of T cells. Recognition may be determined by measuring the level of T cell activation in the presence of a TCR disclosed herein and target cells. Minimal recognition of antigen negative target cells is defined as a level of T cell activation of less than 20%, preferably less than 10%, preferably less than 5%, and more preferably less than 1%, of the level produced in the presence of antigen positive target cells, when measured under the same conditions and at a therapeutically relevant TCR concentration.

For soluble TCRs, a therapeutically relevant concentration may be defined as a TCR concentration of 10^{˜9} M or below, and/or a concentration of up to 100, preferably up to 1000, fold greater than the corresponding EC₅₀ value. Antigen positive cells may be obtained by peptide-pulsing using a suitable peptide concentration to obtain a low level of antigen presentation (for example, 10⁻⁹ M peptide, as described in Bossi et al. (2013) Oncoimmunol. 1;2 (1 1) :e26840) or, they may naturally present said peptide. Preferably, antigen negative cells are human cells.

As is well-known in the art, TCRs may be subject to post translational modifications. Glycosylation is one such modification, which comprises the covalent attachment of oligosaccharide moieties to defined amino acids in the TCR chain. For example, asparagine residues, or serine/threonine residues are well-known locations for oligosaccharide attachment. The glycosylation status of a particular protein depends on a number of factors, including protein sequence, protein conformation and the availability of certain enzymes. Furthermore, glycosylation status (i.e. oligosaccharide type, covalent linkage and total number of attachments) can influence protein function. Therefore, when producing recombinant proteins, controlling glycosylation is often desirable. Controlled glycosylation has been used to improve antibody-based therapeutics. (Jefferis R., Nat Rev Drug Discov. 2009 Mar;8(3):226-34.). For soluble TCRs disclosed herein, glycosylation may be controlled in vivo, by using particular cell lines for example, or in vitro, by chemical modification. Such modifications are desirable, since glycosylation can improve phamacokinetics, reduce immunogenicity and more closely mimic a native human protein (Sinclair A M and Elliott S., Pharm Sci. 2005 Aug; 94(8): 1626-35).

For administration to patients, a TCR disclosed herein (which may be associated with a detectable label or therapeutic agent or expressed on a transfected T cell) or a cell or population of cells disclosed herein may be provided in a pharmaceutical composition together with one or more pharmaceutically acceptable carriers or excipients.

For example, an excipient can give form or consistency, or act as a diluent. Suitable excipients include, but are not limited to, stabilizing agents, wetting and emulsifying agents, salts for varying osmolarity, encapsulating agents, buffers, and skin penetration enhancers. The compositions can be in any suitable form, for example tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. Such compositions may be prepared by any known method, for example by admixing the active ingredient with the carrier(s) or excipient(s) under sterile conditions.

Suitable formulations for parenteral administration include aqueous solutions of the active compounds in water-soluble form, for example, water-soluble salts. In addition, suspensions of the active compounds as appropriate for oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances that increase the viscosity of the suspension and include, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulation may be used simultaneously to achieve systemic administration.

Suitable formulations for oral administration include hard or soft gelatin capsules, pills, tablets, including coated tablets, elixirs, suspensions, syrups or inhalations and controlled release forms thereof. Generally, these agents are formulated for administration by injection (e.g., intraperitoneally, intravenously, subcutaneously, intramuscularly, etc.), although other forms of administration (e.g., oral, mucosal, etc.) can be also used. Accordingly, agents disclosed herein are preferably combined with pharmaceutically acceptable vehicles such as saline, Ringer's solution, dextrose solution, and the like.

The pharmaceutical compositions can also be formulated so as to provide quick, sustained or delayed release of their active ingredients after administration to the patient by employing procedures known in the art. The physical and chemical characteristics of the compositions disclosed herein may be modified or optimized according to the skill in the art, depending on the mode of administration and the particular disease or disorder to be treated. The compositions may be provided in unit dosage form, a sealed container, or as part of a kit, which may include instructions for use and/or a plurality of unit dosage forms. Suitable dose range for a soluble TCR disclosed herein may be between 25 ng/kg and 50 μg kg. A physician will ultimately determine appropriate dosages to be used.

Where more than one agent is to be administered, the agents may be formulated together in the same formulation or may be formulated into separate pharmaceutical compositions. The separate compositions may be administered concurrently, sequentially or separately.

As used herein, the term “therapeutically effective amount” refers to the total amount of the agent or each active component of the pharmaceutical composition or method that is sufficient to provide patient benefit, i.e., prevention or amelioration of the condition to be treated, a reduction in symptoms, an increase in rate of healing, or a detectable change in the levels of a substance in the treated or surrounding tissue. When applied to an individual active ingredient, administered alone, the term refers to that ingredient alone. When applied to a combination, the term refers to combined amounts of the active ingredients that result in the therapeutic effect, whether administered in concurrently, sequentially or separately.

The agents or compositions may be delivered at intervals ranging from about 24 hours to about 2 days, to about 1 week, to about 2 weeks, to about 3 weeks to about 1 month to about 2 months, to about 3 months, to about 4 months, to about 5 months, to about 6 months, to about 12 months, or more. The scheduling of such dosage regimens can be optimized by the practitioner.

The agents or compositions may be administered using a treatment regimen comprising one or more doses, wherein the treatment regimen is administered over 2 days, 3 days, 4 days, 5 days, 6 days or 7 days, 14 days, 30 days, 1 month, 2 months, 3 months, 6 months, 12 months or more.

The term “patient” or “subject,” as used interchangeably herein, refers to any mammal, preferably a human.

The method of any aspect of the invention may be in vivo, ex vivo or in vitro.

The skilled person will appreciate that preferred features of any one embodiment and/or aspect of the invention may be applied to all other embodiments and/or aspects of the invention.

### BRIEF DESCRIPTION OF THE DRAWINGS

FIG. 1—Priming and cloning of HLA-E restricted RL9HIV-specific CD8⁺ T cells from HIV naïve donors. (A) PBMCs from 9 HIV-1 seronegative donors (HD 1 to 9) were stimulated with autologous activated dendritic cells and the RL9HIV peptide for 9 days. HLA-E restricted RL9HIV specific CD8+ T cells were identified using HLA-E-RL9 disulphide trapped tetramer (RL9HIV-(D)) for donors HD1 to 6 or (B) a HLA-E RL9 tetramer generated by UV exchange RL9HIV (UV)) for donors HD 7 to 9. The disulphide was introduced by mutating position 84 of HLA-E heavy chain and adding a glycine and cysteine at the C terminal end of the RL9 peptide in order to stabilize the HLA-E-RL9 complex. (C) disulphide trapped RL9HIV-(D) tetramer+ cells from donor HD1 were sorted for single cell cloning, and positive clones were identified using disulphide trapped RL9HIV-(D)tetramer. (D) RL9 positive clones were CD94 negative and do not recognize HLA-E bound to canonical VL9 signal peptide as illustrated by lack of dual staining with RL9 and VL9 disulphide trapped HLA-E tetramers.

FIG. 2—shows functional analysis of HLA-E restricted RL9^{HIV}-specific CD8⁺ T cell clones. A) IFN-γ, TNF-α, CD107a/b and CD137 expression by 15 RL9 clones upon stimulation with K562 cells transduced with single chain trimer (SCT) disulphide trapped HLA-D-RL9 were assessed using flow cytometry-based readouts; responsive clones could be detected using multiple functional readouts. (B) Six positive clones were further assessed with comparison of stimulation with K562 transduced with HLA-E and pulsed with RL9 peptide, K562 transduced with SCT linked HLA-E RL9 (K562-E-RL9), K562 transduced with SCT disulphide-linked HLA-E RL9 (K562-D-RL9), and K562 transduced with SCT disulphide linked HLA-E VL9 (K562-D-VL9) as a negative control. Horizontal lines indicated means. Groups were analyzed by 2-way ANOVA with Tukey's multiple comparisons. Data shown are representative of six donors and two independent experiments. (C) Blockade of TNF-α secretion and CD107 expression of clone p13c7 by addition of the VL9 peptide prior stimulation with HLA-E transduced K562 cells pulsed with RL9 peptide. Data shown are from two independent experiments.

FIG. 3—shows the recognition of naturally presented RL9 epitope on HIV-1 virus infected cells and elimination of HIV-1-infected targets by RL9 clones. (A) 771.221 cell lines transfected with CD4 were infected with HIV-1NL4.3 virus and cultured alone or with RL9-specific CD8+ T cell clones at an E: T ratio of 1:1. Frequencies of HIV-infected cells (Gag p24+) and the percentage reduction in the frequency of Gag p24+ cells in the presence of the RL9 clones after 5 days of culture (calculated as described in Materials and Methods) are shown. (B) Significant (p=0.031) higher expression of the CD137 activation marker was observed on clone CD8+ T cell clones when cocultured with HIV-1NL4.3 virus infected CD4.221 compared to un-infected CD4.221 cells, and this was significantly blocked by the VL9 peptide (p=0.031). A control EBV-specific CD8+ T cell clone generated from the same donor showed minimal CD137 expression that was not affected by VL9 addition. Horizontal lines indicated means. Data were analyzed by the non-parametric Wilcoxon signed rank test. (C) Purified autologous CD4+ T cells were stimulated with anti-CD3 for 3 days prior HIV-1NL4.3 infection, then either cultured alone or with clones at E: T ratios of 1:1 and 5:1 for 5 days. Gag p24+ cells were gated on CD3+/CD8−/CD4+ and CD4− T cells. A reduction in the proportion of HIV-1NL4.3 infected primary CD4+ T cells was observed at an E: T ratio of 5:1.

FIG. 4—demonstrates the specificity and function of RL9-specific TCRs transduced into primary CD8+ T cells. (A) Two RL9 TCRs p9c1 and p13c7 were transduced into primary CD8+ T cells. CD8+ transductants were stained with disulphide trapped RL9HIV-(D) tetramer initially, washed with PBS, and then stained with anti-mouse TCR Vβ antibody, anti-CD8 and Live/Dead Fixable Aqua. (B) A reduction in the percentage of HIV-1NL4.3-infected primary CD4+ T cells was demonstrated when CD8+ transductants were co-cultured with targets at an E:T ratio of 1:1, with greater reduction observed at an E:T ratio of 5:1. Gag p24+ cells were gated on CD3+/CD8−/CD4+ and CD4− T cells. Bars indicate mean reduction in % of p24+ cells. CD8+ T cells transduced with an irrelevant TCR (IG4) were included as a negative control and CD8+ T cells transduced with a TCR recognizing the B*2705 restricted Gag KK10 epitope were included as a positive control when CD4+ T cells from B*2705+ donor were infected with HIV-1NL4.3 virus and used as targets. Statistical analysis of the data was performed by One-way ANOVA Kruskal-Wallis test. Data shown are representative of four independent experiments. The two experiment where KK10 specific TCR transduced CD8+ T cells were tested on HLA B*2705 negative cells and showed no inhibition are not shown. Primary CD8+ T cells transduced with RL9 TCRs are activated by RL9 stimulation and can reduce the proportion of HIV-infected CD4+ T cells. (C) CD8+ transductants were activated by exposure to autologous B cells pulsed with RL9 peptide or (D) HIVNL4.3-infected CD4.221 cells, indicated by TNFα secretion and up-regulation of CD137. Responses were partially blocked by competitive inhibition with the signal peptide VL9. Gag p24+ cells were gated on CD3+CD8− T cells. Horizontal lines indicated means. Error bars indicated SD. Data shown is representative of three independent experiments.

FIG. 5—demonstrates HIV-1 suppression for two example clones Rev33 and Rev152. Clones were compared with T cell clones specific for a SARSCoV-2 peptide and a T cell clone from the same blood donor but of unknown specificity, that did not bind to the HLA-E Rev tetramer. HIV replication was assessed by intra-cellular staining with anti-HIVp24 antibody (x axis). HIV-1 infected cells also down-regulate surface expression of CD4 (y axis).

### MATERIALS AND METHODS

### Peptides

Synthetic 9 amino acid RL9HIV (RMYSPTSIL) and 11 amino acids RL9HIV-Gly-Cys (RMYSPTSILGC) (SEQ ID NO: 106) peptides were generated by Fmoc (9-fluorenylmethoxy carbonyl) chemistry to a purity of 85% (Genscript, Hong Kong). All peptides were provided as lyophilised power. Following reconstitution to a final concentration of 200 mM in DMSO, peptide stocks were aliquoted and stored at −80° C. until required. A UV photolabile HLA-B leader-sequence peptide (VMAPRTLVL)(SEQ ID NO: 107) incorporating a UV-sensitive 3-amino-3-(2-nitrophenyl)-propionic acid residue (J residue) substitution at the peptide p5 Arg residue was synthesized by Dris Elatmioui at LUMC. This peptide, known herein as the 7MT2 peptide, was stored as lyophilised power at −80° C. and reconstituted as required.

### PCR-Based Site Directed Mutagenesis of the HLA-E*01:03 Heavy Chain

Position 84 Tyr to Cys mutagenesis of the HLA-E*01:03 heavy chain was performed by QuikChange II XL Site-Directed Mutagenesis Kit (Agilent, USA) using the following primers: Fw:5′-CGGACGCTGCGCGGCTGCTACAATCAGAGCGAG-3′ (SEQ ID NO: 107) and Rv:5′-CTCGCTCTGATTGTAGCAGCCGCGCAGCGTCCG-3′ (SEQ ID NO: 108)]. A prokaryotic PET22b+ expression vector encoding HLA-E*01:03 heavy chain (residues 1-276) linked to a 15 amino acid biotinylation AviTAG was used as PCR template. Following mutagenesis and transformation into XL10 Gold bacteria, individual colonies were grown overnight in low salt Luria-Bertani (LB) broth containing 100 μg/mL Carbenicillin. All plasmids were extracted with Spin miniprep kit (Qiagen, UK), and their sequences were confirmed by DNA Sanger sequencing.

### Protein Production

Both canonical and Tyr84Cys mutated HLA-E*01:03 heavy chains were expressed in E. coli BL21(DE3)pLysS competent bacterial cells (Promega, UK). Single colonies inoculated in 1 Litre low salt Luria-Bertani (LB) broth containing 100 μg/mL Carbenicillin were incubated overnight at 37° C. The following day, approximately 75 mL of overnight starting cultures were transferred to fresh 3×1 litre Carbencillin-spiked LB broth. Following incubation to an OD600 of 0.5, protein expression was induced by the addition of 0.5 mM IPTG. The cultures were subsequently incubated for a further 4 hours prior to bacterial pellet recovery via centrifugation at 1000 g for 20 minutes at 4° C. Inclusion body proteins were extracted from bacterial pellets by sonication and homogenisation in a Triton-based buffer (Triton X-100, 50 mM Tris, 100 mM NaCl, 0.1% Sodium Azide, 1 mM EDTA, 1 mM DTT) followed by resuspension in a Tris-NaCl buffer (50 mM Tris, 100 mM NaCl, 1 mM EDTA, 1 mM DTT). HLA-E*01:03 heavy chains were solubilised in 8 M urea containing 50 mM MES pH 6.5, 0.1 mM EDTA, 0.1 mM DTT and subsequently aliquoted at 10 mg/mL and stored at −80° C. until required.

### Protein Refolding and Purification

Conventional and Tyr84Cys mutated HLA-E*01:03 heavy chains were refolded using standard MHC refolding methods 2, 31 but with slight modifications. In brief, β2 m (2 μM final concentration) was refolded in MHC Refold Buffer (100 mM Tris pH8.0, 400 mM L-Arginine monohydrochloride, 2 mM Ethylenediamineteraacetic acid, 5 mM reduced Glutathione and 0.5 mM oxidised Glutathione) for 30 minutes at 4° C. for 30 minutes, following which either 30 μM RL9HIV-GC peptide (for Cys trapped refolds) or 50 μM RL9HIV peptide (for conventional refolds) was added. Conventional or Tyr84Cys mutated HLA-E*01:03 heavy chains were subsequently pulsed into the refolding buffers respectively, up to a final concentration of 1 μM. After 72 hours, all refolds were filtered using 22 μm cellular nitrate membrane (GE Healthcare, UK) before concentration using Vivaflow 50 (Sartorius, Germany) and Ultra-15 10-kDa cut-off centrifugal units (Sartorius, UK). The samples were subsequently buffered exchanged, using Sephadex G-25 PD10 columns (GE Healthcare, UK) into 10 mM Tris for overnight AviTAG biotinylation using the BirA enzyme (Avidity, USA) according to the manufacturer's instructions. Correctly refolded complexes were purified by size exclusion fast protein liquid chromatography (FPLC) into 20 mM Tris pH8 and 100 mM NaCl buffer using a HiLoad 16/600 Superdex 75 pg column. Correctly folded β2m-HLA-E*01:03-peptide complexes were retrieved, concentrated to 2 mg/mL and snap frozen for subsequent tetramer generation.

### Generation of UV Exchange RL9-Loaded HLA-E:

Refolding of the VL9-based UV sensitive (7MT2) peptide with HLA-E and β2m was performed. Protein concentration and biotinylation was carried out as per the method described in “Protein refolding and purification”. For UV-mediated peptide exchange reactions, 10 wells comprising 0.5 μM (˜25 mg/mL) of HLA-E-7MT2 monomer were incubated with 150 μM RL9 peptide in polypropylene V-shaped 96-well plates (Greiner Bio-One, Austria). UV exchange buffer (20 mM Tris, pH 7.4, 150 mM NaCl) was added to each well to adjust the final reaction volumes to 125 μL. UV exchange samples were incubated under a Camag UV cabinet with a long-wave 366 nm UV lamp for 60 minutes on ice. Following photo-illumination, the samples were centrifuged at 4000 g for 20 minutes to remove aggregated material. Aggregate-cleared samples were pooled and conjugated to fluorescent dyes as described below (Tetramer generation and staining protocol).

### Tetramer Generation Protocol

Disulphide linked and UV-peptide exchange HLA-E*01:03-RL9 tetramers were generated via conjugation to streptavidin-bound APC (Biolegend, San Diego) or BV421 (Biolegend, San Diego) at a Molar ratio of 4:1.

Transduction of K562 Cell Line with HLA-E-RL9 Single Chain Trimers (SCT) Construct (K562-E-RL9) and Disulphide Linked Construct (K562-D-RL9)

Single chain trimers (SCT) of HLA-E*01:03 with the RL9HIV peptide (RMYSPTSIL) constructs were generated. A disulphide “trap” was engineered into the SCT by mutating position 84 of HLA-E to cysteine and changing the sequence of the first flexible linker (between the peptide and beta2-microglobulin) to GCGGSGGGGSGGGGS (SEQ ID NO: 109). Plasmid constructs were generated with HLA-E-E-RL9 or HLA-E-D-RL9 into the retrovirus vector, pMSCV-GFP (Cell Biolabs). Retroviral particles were produced by mixing 2 μg of the HLA-E plasmids with 0.5 μg of pCMV-VSV-G (Cell Biolabs) and 200 μl of OPTI-MEM (Gibco) for at room temperature. 7 μl of X-tremeGENE HP Transfection reagent (Roche) was added and incubated at 37° C., 5% CO2 for 15 mins. This transfection solution was added to PlatGP cells (Cell Biolabs) and incubated overnight at 37° C., 5% CO2.

Retroviral particles were harvested after 24 hours and stored for 3 days after initial transfection. Twenty-four well plates pre-coated with 15 μg/ml RetroNectin were blocked with 2% BSA, PBS. 1×10⁶ K562 cells were transduced in each well with 2 ml of retrovirus supernatant by centrifugation at 100 g, for 2 hours at 32° C. HLA-E transduced K562 cells were further purified by cell sorting on the expression of HLA-E as determined by staining with the W6/32 mAb clone (BioLegend).

### TCR Sequencing

RNA was extracted from the T cell clones using an RNeasy Micro Kit (Qiagen), following manufacturer's instructions. TCR cDNA was generated by template-switch reverse transcription, using a template switch oligo, and primers specific to the constant regions of Trac and Trbc genes, and SMARTScribe Reverse Transcriptase (Takara). TCR DNA was amplified by two subsequent rounds of nested PCR using Phusion High-Fidelity PCR Master Mix (NEB). One last PCR was performed to add the Illumina adaptors and indexes. TCR libraries were sequenced using an Illumina Miseq Reagent Kit V2 300-cycle on the Illumina Miseq platform. FASTQ files were demultiplexed and TCR sequences analysed using MiXCR software [doi:10.1038/nmeth.3364]. Post analysis was performed using VDJ tools [doi:10.1371/journal.pcbi.1004503].

TCR Transductions into Primary CD8+ T Cells

TCR alpha and beta VDJ regions were amplified by PCR from the DNA generated during the preparation of TCR sequencing libraries. These products were assembled into a pHR-SIN backbone with the murine TCR alpha and beta constant regions, using the HiFi DNA Assembly cloning kit (NEB). Correct plasmid sequences were confirmed by Sanger sequencing. Lentiviruses were produced by transfecting the TCR-containing plasmid plus pMDG-VSVG, and pCMV-dR8.91 packaging plasmids into HEK 293T cells, using the transfection reagent TurboFectin (Origene). Lentiviral supernatants were collected 48 h after transfection, centrifuged at 2000 rpm to remove cellular debris and transferred to Retronectin (Takara Bio) treated 48-well plates. The plates were centrifuged 1.5 h, at 2000× g to facilitate virus binding and supernatant was subsequently removed. Primary T cells were isolated from PBMC by positive selection using MACS beads (Miltenyi) and activated for 2 days with 1:1 CD3/CD28 Dynabeads (Thermo Fisher) in RPMI medium supplemented with 1% non-essential amino acids, 1% sodium pyruvate, 1% glutamine, 1% HEPES, 1% pen-strep 0.1% β-mercaptoethanol (Invitrogen), 5% pooled AB human sera (UK National Blood Service), 500 U/mL IL-2 (University of Oxford), and 10 ng/mL rhlL-5 (Peprotech). Activated T cells were transferred to lentivirus-coated plates at 0.25×10⁶ cells/mL and cultured for 4 days. Mouse TCRβ+ CD8+ tetramer+ cells were purified by flow cytometry (BD Fusion) and expanded for extra 17 days before usage in subsequent assays.

### Constructing of HLA-B27:05-Restricted HIV Gag₂₆₃₋₂₇₂ (KK10) TCR CD8+ T Cell Transductants

CD8+ T cell transductants targeting the HIV-1 Gag₂₆₃₋₂₇₂ KK10 epitope (KRWIILGLNK) restricted by HLA-B*27:05 were based on the published C12C clone (Ladell K, et al. 2013. A Molecular Basis for the Control of Preimmune Escape Variants by HIV-Specific CD8⁺ T Cells. Immunity 38(3):425-436). To construct this full-length TCR construct, the published C12C CDR3α and CDR3β sequences were utilised and combined these with nucleotide sequence provided by IMGT for TRBV6-5, TRBJ1-1, TRAV14, and TRAJ21. After murinization and cysteine modification of the constant domains of the TCR and insertion of an additional cysteine bridge, the complete sequence of both TCR chains was constructed with a 2A sequence for bi-cistronic expression (Genscript, Piscataway Township, NJ, USA) and cloned into the pMP71 backbone. To produce retroviral supernatants, the TCR construct was transfected into the embryonal kidney cell line 293Vec-RD114 (BioVec Pharma, Québec, Canada). Collected supernants were then purified via centrifugation on a 20% sucrose gradient. Viral transduction of activated CD8+ T cells was done by magnetofection using Viromag Viral Transduction reagent (Oz Biosciences, Marseilles, France) according to manufacturer's protocol. CD8+ T cell transductants were then cultured in X-vivo 15 (Lonza, Basel, Switzerland) supplemented with 10% FBS and 200 U/ml IL-2 until use in assays.

### Cell Lines and Primary Cells

MHC-I null cell line K562 transfected with HLA-E*01:03 (K562-E line) was generously provided by Thorbald van Hall (Leiden University Medical Center). The 721.221 HLA-class I deficient cell line transfected with CD4 (721.221-CD4) was generously provided by Masafumi Takiguchi, Univeristy of Kumomoto, Japan. PBMCs were isolated from HIV negative donor leukapheresis cones (NHS Blood Transfusion Services, Bristol, UK) by density gradient separation. CD4+ and CD8+ T-cells were enriched from PBMC by positive selection using magnetic bead according to manufacturer's instructions (MACS, Miltenyi Biotech, Surrey, UK).

### Tetramer Staining Protocol

Cells were stained with disulphide linked HLA-E-RL9 tetramer or UV exchanged RL9 tetramer both conjugated with APC at 0.5 μg per 1×10⁶ cells in 100 μl MACs buffer (PBS with 2 mM EDTA and 0.5% BSA) at room temperature (RT) for 45 minutes in the dark. After the PBS wash, cells were further stained with cell surface antibody CD8-BV421 (BioLegend) and the Live/Dead Fixable Aqua (Thermo Fisher Scientific) in 100 μl PBS for 30 min at RT in the dark. After the PBS wash and fixed with 2% paraformaldehyde, cells were acquired using an LSR Fortessa (BD Biosciences) and analysed using FlowJo software v10.3 (Tree Star).

### In Vitro Priming of HLA-E Restricted RL9HIV Specific CD8+ T Cells

On day 0, 100 to 150×10⁶ freshly isolated PBMCs were plated at 10⁷/ml in 6-well plates in AIM-V medium (Invitrogen) with dendritic cell (DC) differentiation cytokine cocktail of GM-CSF (1000 U/ml, Miltenyi Biotech Ltd) and IL-4 (500 U/ml, Miltenyi Biotech Ltd). On day 1, DC maturation stimuli of TNF-α (1000 U/ml, R&D Systems), IL-1β (10 ng/ml, R&D Systems) and prostaglandin E2 (PGE2 1 μM, Merck) were added together with RL9HIV peptide (20 μM, GenScript), IL-7 (5 ng/ml, R&D Systems) and IL-15 (5 ng/ml, R&D Systems). On day 6, IL-2 was added at a concentration of 500 IU/ml (University of Oxford). Cells were ready for HLA-E RL9 tetramer analysis on day 9. In selected experiments, cells were further stimulated with irradiated K562-D-RL9 cells for 7 days.

### Cloning of HLA-E Restricted RL9HIV Specific CD8+ T Cells

After RL9HIV priming, PBMCs were stained with APC conjugated disulphide linked HLA-E RL9 tetramer at 5 ug per 5×10⁷ cells in 500 μl MACs buffer at RT for 45 minutes in the dark. After the PBS wash, cells were further stained with anti-CD3-APC-Cy7, anti-CD4-PerCP-Cy5.5, anti-CD8-BV421, anti-CD94-FITC (All BioLegend) and the dump markers Live/Dead Fixable Aqua, anti-CD56-BV510 (BD Biosciences) for 30 min at RT in the dark. Tetramer+/CD3+/CD8+/CD4−/CD56−/CD94−/live subsets were sorted using a FACS Aria III (BD Biosciences).

Sorted tetramer+ cells were seeded at 0.4 cells/well into 384-well plates (Corning) with phytohemagglutinin (PHA 1 mg/mL, Remel) and irradiated (45 Gy) allogeneic feeder cells from 3 different healthy blood cones (10⁶ feeder cells/mL) in RPMI 1640 glutamine [-] medium (Invitrogen) supplemented with non-essential amino acids (1%, Invitrogen), sodium pyruvate (1%, Invitrogen), glutamine (1%, Invitrogen), b-mercaptoethanol (0.1%, Invitrogen), penicillin/streptomycin (1%, Invitrogen) (RPMI 1640 complete media (RPMI 1640 CM)) with pooled AB human sera (10%, UK National Blood Service) and IL-2 (500 U/mL, University of Oxford). After 10 days, T cell clones were identified and transferred into 96-well round-bottom plates (Corning). An aliquot of each clone was stained with HLA-E-RL9 disulphide linked tetramer and anti-CD3-APC-Cy7, anti-CD8-BV421, anti-CD4-PerCP -Cy5.5 anti-CD94-FITC antibodies and dump markers Live/Dead Fixable Aqua, anti-CD56-BV510 to confirm RL9HIV specificity.

### Intracellular Staining of IFN-γ, TNF-α, CD107a/b and CD137

Clone cells were washed and left in fresh RPMI 1640 CM (5% AB serum) without IL-2 to rest for 5 hours or overnight before being stimulated with RL9 peptide pulsed K562-E (5004, 20-24 hours at 27° C.), K562-E-RL9 or K562-D-RL9 cells at clone:stimuli cells ratio of 1:3 for 1 hour, followed by addition of 5 μg/ml Brefeldin A (Biolegend) and 5 μg/ml GolgiStop (BD Biosciences) for an additional 8 hours at 37° C. For CD107 staining, anti-CD107a-BV421 and anti-CD107b-BV421 (Biolegend) antibodies were added at beginning of co-culture. After 9 hours incubation, cells were washed with PBS and stained with Live/Dead Fixable Aqua, anti-CD8-PerCP-Cy5.5 and anti-CD3-APC-Cy7 for 30 min at RT first, then fixed/permeabilized with Cytofix/Cytoperm 1× Solution (BD Biosciences) for 10 min at 4° C., and stained in Permwash 1× Solution (BD Biosciences) with anti-TNFα-PE, anti-IFN-γ-FITC and anti-CD137-BV650 (All BioLegend) for 30 min at RT. After being washed with PBS and fixed with 2% paraformaldehyde, samples were acquired using an LSR Fortessa (BD Biosciences) and analysed using FlowJo software v10.3 (Tree Star). In the selected assays to determine HLA-E restriction, K562-E cells were pre-incubated with VL9 canonical signal peptide (VMAPRTLVL, 50 μM, 3 hours at 27° C.) prior to addition of RL9 peptide.

### Activation of RL9TCR Transductants

Primary CD8+ transductants were washed and rested in RPMI 1640 CM media with 10% human serum for minimal 5 hours or overnight prior stimulated with RL9HIV peptide pulsed autologous B cells (50 μM, 2 hours at 37° C.) at transductants:B cells ratio of 1:2 for intracellular TNFαcytokine and CD137 staining as described in “Intracellular staining of IFN-γ, TNF-α, CD107a/b and CD137”.

### Viral Inhibition/Infected Cell Elimination Assay (VIA)

PBMCs were stimulated with anti-human CD3 at 100 ng/ml (clone OKT3, TONBO Biosciences) in RPMI 1640 CM supplemented with 5% AB human serum and IL-2 (100 IU/ml) for 5 days. CD4+ cells were enriched from activated PBMC by positive selections using anti-CD4 magnetic beads according to the manufacturer's instructions (MACS, Miltenyi Biotech, Surrey, UK). Activated CD4+ cells or 721.221-CD4 cells were infected with the HIVNL4.3 virus obtained from the Programme EVA Centre for AIDS Reagents (National Institute for Biological Standards and Control (NIBSC), a centre of the Health Protection Agency, UK.) at a multiplicity of infection of 1×10⁻² by spinoculation for 2 hours at 27° C,. HIV NL4.3-infected target cells (primary CD4+ T-cells or 721.221-CD4 cells) were washed with RPMI 1640 CM and cultured in triplicate (1×10⁵ cells/well) in RPMI 1640 CM supplemented with 5% AB serum and IL-2 (50 IU/ml), either alone or with RL9 clone cells or primary CD8+ transductants for 5 days at various Effector:Target (E:T) ratios. An EBV clone (B*0801 restricted RAKFKQLL specific) or non-transduced primary CD8+T cells were used as a control for RL9 specificity. At end of 5 days' coculture, cells were collected and stained with Live/Dead Fixable Aqua before permeabilised with BD fix/perm solution for intracellular HIV gag p24 (Beckman Coulter, UK) staining followed by anti-CD3-APC-Cy7, anti-CD8-BV421, anti-CD4-PerCP-Cy5.5 antibodies. The frequency of infected cells was determined by intracellular staining for Gag p24 Ag, optimized for sensitivity and specificity. To demonstrate the presentation of HLA-E bound RL9 epitopes on HIV NL4.3 infected CD4+ T cells, selected experiments were conducted with the addition of VL9 canonical signal peptide (50 μM) in the coculture of targets and effectors.

Viral inhibition/infected cell elimination was calculated by normalising to data obtained with no effectors using the formula: (fraction of Gag+ cells in CD4+ T-cells cultured alone—fraction of Gag+ in CD4+ T-cells cultured with CD8+ clone cells)/fraction of p24+ cells in CD4+ T-cells cultured alone×100%. In selected experiments, CD8+ clone T-cells were analysed for expression of activation markers CD137 using BV421-conjugated antibodies (BD Biosciences) at 24 hours post effector and target co-culture.

The VIA was set up with a minimum of 3 replicates for each culture condition. Cells from each culture condition were harvested and pooled for intracellular p24 staining to reach the required acquisition of at least 10000 viable target cells of each target and effector coculture.

### Statistical Analysis

Statistical analysis was performed using GraphPad Prism software (version 6.0 or later). Data with skewed distribution were analysed with the non-parametric test, Wilcoxon signed rank test. Where a normal distribution was assumed, data were analysed with parametric tests (Repeated Measures 2-way ANOVA with Tukey's multiple comparisons tests).

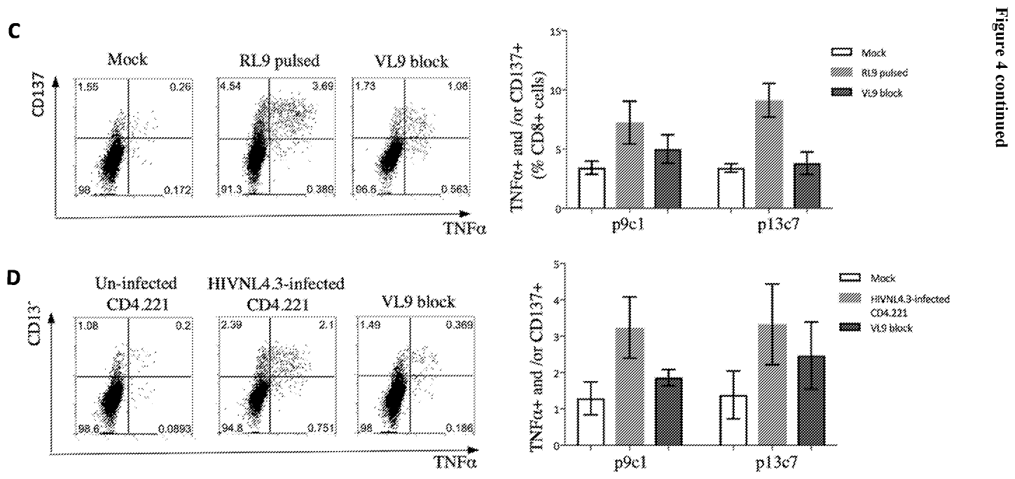

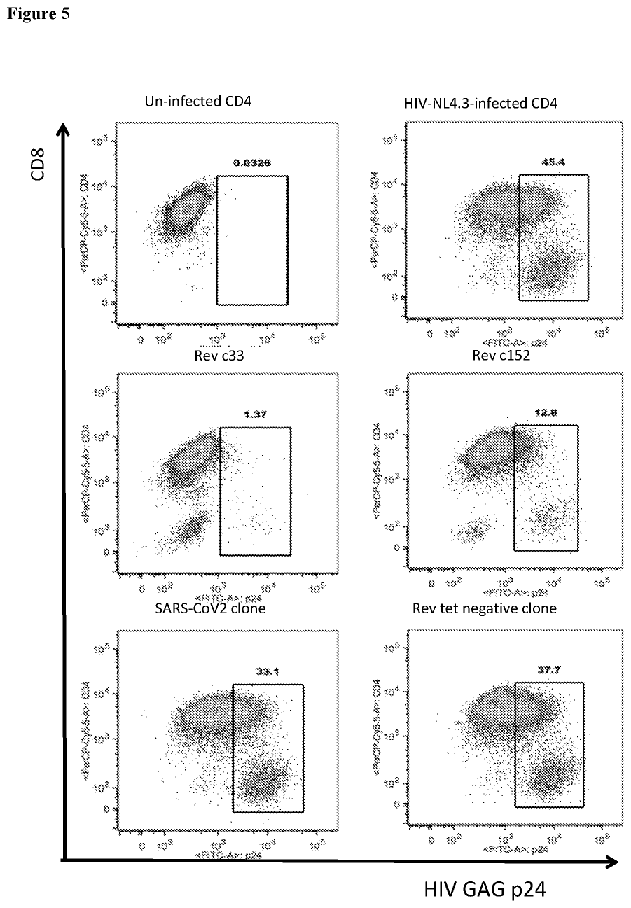

All methods outlined above were also used to generate data for FIG. 5 and Table 2, relating to IL9 and TCRs which recognise HLA-E bound IL9.

TABLE 1
T cell receptor (TCR) sequences of HLA-E restricted RL9HIV-specific
CD8+ T cell clones

| clones | V | J | CD3 alpha | V | J | CD3 beta |
|---|---|---|---|---|---|---|
| P4c2 | TRAV38- | TRAJ28 | CAFMKLHS | TRBV12- | TRBJ1-2 | CASSLWAVG |
| | 1 | | GAGSYQLT | 4 | | YGYTF |
| | | | F | | | |
| P8c6 | TRAV38- | TRAJ43 | CAFDNNND | TRBV5-6 | TRBJ1-1 | CASSLVGAIT |
| | 1 | | MRF | | | EAFF |
| p8c7A | TRAV38- | TRAJ28 | CAFVDGAG | TRBV9 | TRBJ1-6 | CASSVGNSN |
| | 1 | | SYQLTF | | | SPLHF |
| p8c7B | TRAV23 | TRAJ57 | CAASGLFIQ | TRBV9 | TRBJ1-6 | CASSVGNSN |
| | DV6 | | GGSEKLVF | | | SPLHF |
| P8c8 | TRAV12- | TRAJ48 | CAVYGSGK | TRBV28 | TRBJ2-6 | CASSFGPSSG |
| | 2 | | LTF | | | ANVLTF |
| P9c1 | TRAV38- | TRAJ45 | CAFTLYSG | TRBV28 | TRBJ2-3 | CASSLPTSLS |
| | 1 | | GGADGLTF | | | TDTQYF |
| P9c5 | TRAV12- | TRAJ29 | CAMSWNS | TRBV2 | TRBJ1-3 | CASSVTGVR |
| | 3 | | GNTPLVF | | | NTIYF |
| P11c5 | TRAV13- | TRAJ16 | CAAYGQKL | TRBV28 | TRBJ2-2 | CASSLLEPDL |
| | 1 | | LF | | | NTGELFF |
| P12c6 | TRAV1-2 | TRAJ34 | CAVNTDKL | TRBV7-9 | TRBJ2-3 | CASSNPGNS |
| | | | IF | | | DF |
| P13c2 | TRAV29 | TRAJ22 | CAAVSTGS | TRBV5-1 | TRBJ2-6 | CASSLAKGA |
| | DV5 | | ARQLTF | | | NVLTF |
| P13c7 | TRAV10 | TRAJ17 | CVVSAWDP | TRBV6-6 | TRBJ2-3 | CASSPGGQG |
| | | | AAGNKLTF | | | LDTQYF |
| P14c3 | TRAV38- | TRAJ43 | CAYNRND | TRBV19 | TRBJ1-5 | CASSTDRDN |
| | 2DV8 | | MRF | | | QPQHF |
| p16c5 | TRAV19 | TRAJ40 | CALSEALTS | TRBV20 | TRBJ2-7 | CSASVGKSS |
| | | | GTYKYIF | | | YEQYV |

TABLE 2
T cell receptor (TCR) sequences of HLA-E restricted IL9HIV-specific
CD8+ T cell clones

| clones | V | J | CD3 alpha | V | J | CD3 beta |
|---|---|---|---|---|---|---|
| Rev33 | TRAV41 | TRAJ57 | CAVLLITQ | TRBV9 | TRBJ2-3 | CASSVGGTN |
| | | | GGSEKLVF | | | TQYF |
| Rev141 | TRAV1- | TRAJ31 | CAVRDED | TRBV20- | TRBJ1-2 | CSARGGGN |
| | 2 | | ARLMF | 1 | | RESHYGYT |
| | | | | | | F |
| Rev152 | TRAV35 | TRAJ45 | CAGQYSG | TRBV5-6 | TRBJ2-5 | CASSLPDSS |
| | | | GGADGLT | | | ETQYF |
| | | | F | | | |
| Rev160 | TRAV19 | TRAJ32 | CALSEYG | TRBV15 | TRBJ1-1 | CATSRFLEG |
| | | | GATNKLIF | | | KDTEAFF |
| Rev164 | TRAV41 | TRAJ45 | CAGGGGA | TRBV29- | TRBJ1-1 | CSVAETGT |
| | | | DGLTF | 1 | | EAFF |
| Rev166 | TRAV19 | TRAJ28 | CALSEAYS | TRBV19 | TRBJ1-5 | CASNVGEG |
| | | | GAGSYQL | | | YNQPQHF |
| | | | TF | | | |
| Rev169 | TRAV41 | TRAJ48 | CAAWAPT | TRBV9 | TRBJ2-2 | CASSVGYP |
| | | | NFGNEKL | | | GELFF |
| | | | TF | | | |
| Rev178 | TRAV13- | TRAJ32 | CAETLTH | TRBV2 | TRBJ2-7 | CASSEPGA |
| | 2 | | GGATNKLI | | | AYEQYF |
| | | | F | | | |
| Rev185 | TRAV25 | TRAJ54 | CKGGAQK | TRBV27 | TRBJ2-7 | CASSLGRSY |
| | | | LVF | | | EQYF |
| Rev189 | TRAV3 | TRAJ21 | CAVRDRN | TRBV28 | TRBJ1-4 | CASSGVKG |
| | | | NFNKFYF | | | TGSEKLFF |
| Rev193 | TRAV14 | TRAJ28 | CAMREGA | TRBV9 | TRBJ1-1 | CASLAGQG |
| | DV4 | | GAGSYQL | | | RSEAFF |
| | | | TF | | | |
| Rev197 | TRAV12- | TRAJ10 | CAVSTGG | TRBV2 | TRBJ2-7 | CASSDGLR |
| | 2 | | GNKLTF | | | GSVRYEQY |
| | | | | | | F |
| Rev198 | TRAV39 | TRAJ31 | CAVVWAT | TRBV10- | TRBJ1-2 | CAIGGQEIL |
| | | | RLMF | 3 | | MHGYTF |
| Rev203 | TRAV29 | TRAJ22 | CAATFVS | TRBV9 | TRBJ2-3 | CASSLRRA |
| | DV5 | | GSARQLTF | | | HTDTQYF |
| Rev204 | TRAV19 | TRAJ11 | CALSRRYS | TRBV6-6 | TRBJ2-7 | CASRLTDS |
| | | | TLTF | | | YEQYF |
| Rev221 | TRAV21 | TRAJ37 | CAAGSSN | TRBV29- | TRBJ2-7 | CSVEFASK |
| | | | TGKLIF | 1 | | GYEQYF |
| Rev226 | TRAV23 | TRAJ30 | CAATPLLG | TRBV27 | TRBJ1-1 | CASSFRGEA |
| | DV6 | | ADKIIF | | | EAFF |
| Rev229 | TRAV21 | TRAJ28 | CAGSGYS | TRBV27 | TRBJ2-3 | CASSFSAGT |
| | | | GAGSYQL | | | DTQYF |
| | | | TF | | | |
| Rev230 | TRAV21 | TRAJ57 | CAVSSQV | TRBV28 | TRBJ1-2 | CASSPPTLG |
| | | | TGGSEKL | | | YGYTF |
| | | | VF | | | |

### EXAMPLES

### Example 1—Cloning and Priming of RL9 Specific CD8 T Cells

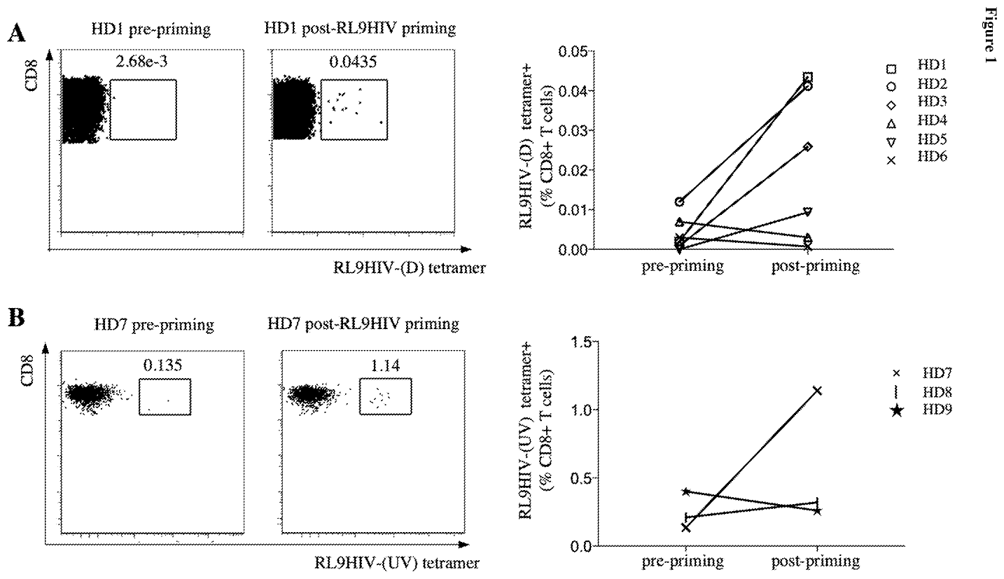

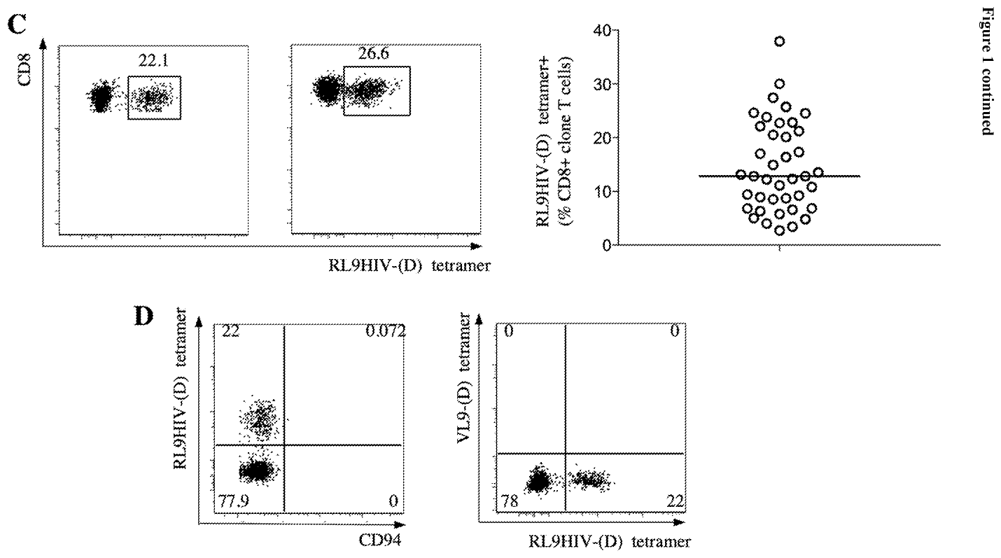

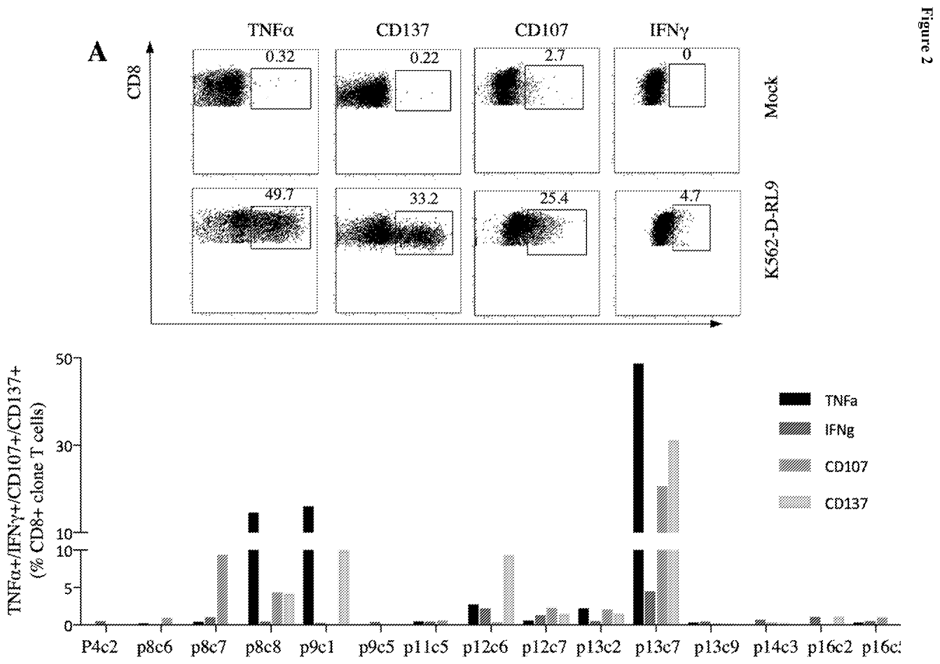

HLA-E-RL9 disulphide trapped tetramer was used to stain CD8+ PBMC from six HIV-1 negative blood donors (FIG. 1a). HLA-A2 negative donors were chosen as some peptide binding motifs previously described for HLA-E also overlap with those reported for HLA-A2 (M. H. Lampen et al., Alternative peptide repertoire of HLA-E reveals a binding motif that is strikingly similar to HLA-A2. Mol Immunol 53, 126-131 (2013).

Initial tetramer staining showed cells that bound to tetramer were very rare in freshly isolated PBMCs with a mean value of 0.004% of CD8+ T cells stained for HLA-E-RL9 disulphide trapped tetramer (FIG. 1a). To expand epitope-specific T cells, total PBMCs were cultured for 9 days with a dendritic cell differentiation cytokine cocktail of GM-CSF and IL-4, with addition of the DC maturation stimuli of TNF-α, IL-1β and prostaglandin E2 after 1 day together with the RL9 peptide, IL-7 and IL-15 to prime RL9 specific CD8+ T cells, and of IL-2 on day 6 ²². After 9 days, cell expansion was monitored using HLA-E-RL9 disulphide trapped tetramer. As shown in FIG. 1a; tetramer reactive T cells were clearly detectable, although still rare (mean 0.019% of CD8+ T cells). In one selected donor HD1, with the best tetramer-reactive T cell frequencies after expansion, T cells were further stimulated with irradiated K562 cells expressing a disulphide trapped single chain trimer of HLA-E-β2m-RL9 for a further 7 days, prior to further monitoring using the HLA-E-RL9 disulphide trapped tetramer. This approach achieved limited further expansion of positive cells (data not shown). The latter PBMCs were then sorted by HLA-E-RL9 disulphide trapped tetramer and plated out at 0.4 cells per well in microtiter wells along with PHA and IL-2. After two weeks, 171 clones had grown, of which 40 had >2% of cells stained positive with the disulphide trapped RL9 tetramer (FIG. 1c). All RL9 positive clones were CD94 negative [FIG. 1d(i)] and did not stain with tetramers of HLA-E disulphide trapped to the canonical HLA class I signal peptide VL9 [FIG. 1d(ii)]. 15 clones that stained in the tetramer positive range of 10-40% were chosen for further study. When these cells were fixed in 2% paraformaldehyde immediately after tetramer staining but in the absence of washing, close to 100% staining was observed (Supplementary FIG. 1), suggesting that the lower values on unfixed cells were caused by relatively low affinity T cell receptor (TCR) binding. These clones were then maintained in culture for approximately 2 months and expanded with irradiated feeders and PHA every 15-18 days.

In a separate experiment, RL9 specific CD8+ T cells were expanded from PBMC from three additional HIV-1 negative blood donors following the same protocol. For comparison, an HLA-E RL9 non-trapped tetramer was freshly prepared using an UV-mediated peptide exchange refolding method {Walters, 2020 #57}. Here, HLA-E was first refolded stably with the signal VL9 peptide modified to replace position 5 arginine with the light sensitive 3-amino-3- (2-nitrophenyl)-propionic acid residue and then the RL9 peptide was exchanged by exposing this complex to UV light in the presence of excess RL9 peptide.

Staining of PBMCs with the UV exchanged HLA-E RL9 tetramer prior to expansion showed a mean of 0.25% tetramer-positive, a value higher than that observed in the first 6 donors studied due to slightly higher non-specific binding of this tetramer compared to the disulphide trapped HLA-E-RL9 tetramer (FIG. 1b). One donor, HD7 showed a marked increase in the untrapped tetramer positive population after priming (0.2% to 1.14%) (FIG. 1b), However, as these T cells did not expand sufficiently during further rounds of re-stimulation, they were not included for follow-up in this study.

### Example 2—Functional Analysis of HLA-E Restricted T Cell Clones

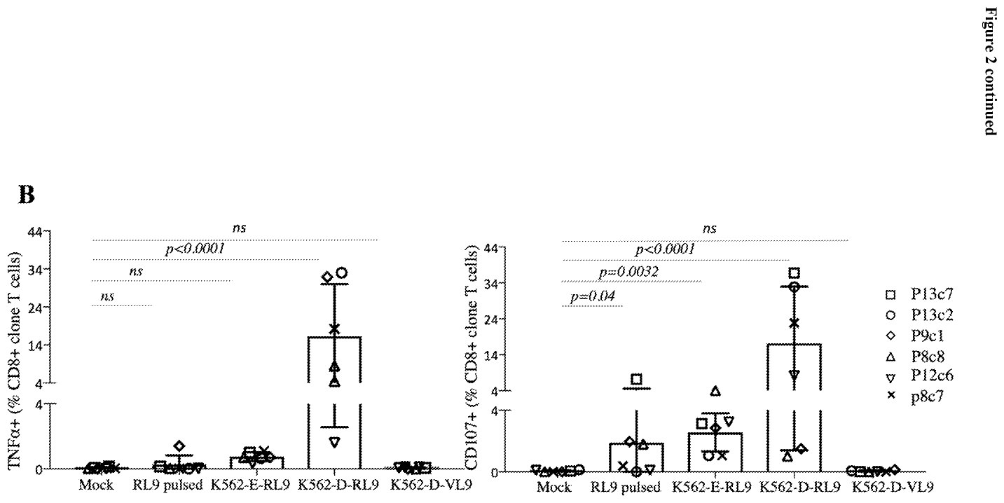

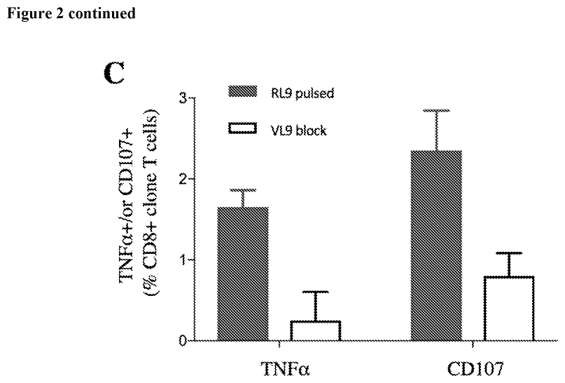

As shown above, all selected CD8+ T cell clones stained positive with the HLA-E-RL9 disulphide trapped tetramer. Their functional capacities were tested by stimulation with (HLA-negative) K562 cells expressing a disulphide trapped single chain trimer of HLA-E-β2m-RL9. As shown in FIG. 2a, this stimulated TNFα, IFNγ secretion and CD107a/b, CD137 expression. These responses were compared to those elicited by K562 cells expressing HLA-E-β2m-RL9 as a single chain trimer, but untrapped ^{2, 30} where peptide was not cross-linked via a HLA-E heavy chain 84C—peptide C bridge, and by K562 cells expressing wild type HLA-E-β2m dimers pulsed with RL9 peptide. Responses to both of these stimuli were much weaker than thoses elicited by cells expressing the disulphide trapped HLA-E-β2m-RL9 single chain trimer [FIG. 2b(left) and (ii)]. CD107a up-regulation was more readily elicited than TNFα production with most clones demonstrating significant responses to RL9 peptide pulsed K562-E cell line and untrapped K562-E-RL9 single chain trimer [FIG. 2b(right)]. One clone, p13c7 gave a measurable response to RL9 peptide pulsed cells. Additionally, TNFα and CD107a/b responses generated by this clone were blocked by competitive inhibition with the canonical HLA-E binding VL9 signal peptide, confirming that RL9 was presented by HLA-E (FIG. 2c).

### Example 3—T Cell Clones Recognise HIV-1 Infected Cells

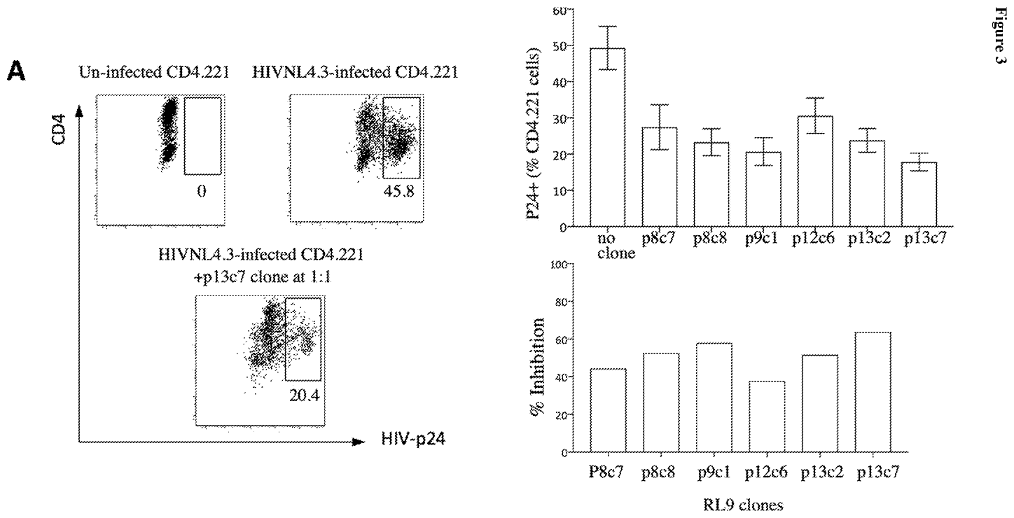

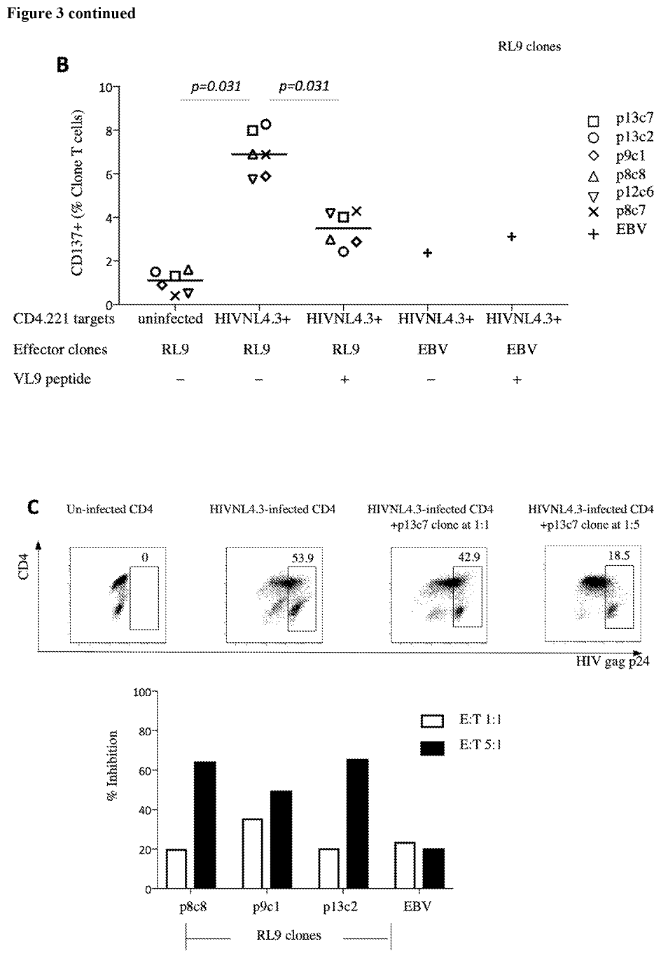

Given that T cells were primed in vitro with the 9mer RL9 peptide and selected primarily for binding to HLA-E-RL9 disulphide trapped tetramers, it was important to check whether they could recognise HIV-1 infected cells. This experiment would test whether the RL9 peptide is processed and presented on infected cells, as would be expected from the analogous observations made with SIV infected cells from rhesus macaques. Therefore a viral inhibition assay (VIA) was set up, as used previously for the classical MHC Ia restricted CD8 T cells ²³, where CD4-expressing 721.221 HLA-Ia negative, but HLA-E positive, cells were infected with HIV-1 NL4.3 and then incubated with the test T cell clone at an E:T ratio of 1:1 for 5 days. HIV-1 Gag p24 expression was measured as an indicator of HIV-1 infection and the reduction of Gag p24+ cells was evaluated as a measure of either inhibition of HIV-1 replication and/or lysis of HIV-1 infected cells, mediated by the T cell clone. Six clones were tested, and were shown to reduce p24 positive cells by 15-45% (FIG. 3a). 721.221-CD4 cells infected with HIV-1 NL4.3 stimulated significantly higher upregulation of surface CD137, a marker of T cell activation^{24, 25} on the T cell clones compared to stimulation by uninfected cells (p=0.031). This activation was blocked by addition of the VL9 signal peptide (p=0.031), indicating that the T cells recognised peptide bound to HLA-E. A control CD8+ T cell clone specific for the Epstein-Barr Virus BZLF1 peptide RAKFKQLL, generated from the same donor as the RL9 specific clones, did not respond to HIV-1 infected 721.221-CD4 cells (FIG. 3b).

As a more physiological target, the VIA was performed on purified autologous CD4+ T cell targets, stimulated with anti-CD3 for 3 days prior HIV-1 NL4.3 infection. Infected CD4+ T cells were cultured alone or in the presence of RL9-reactive clones at E:T ratios of 1:1 and 5:1 for 5 days. Viral inhibition by clones was again observed, with greater suppression obtained at clone to target cell ratios of 5:1 than at 1:1 (FIG. 3c).

### Example 4—TCR Analysis and Construction

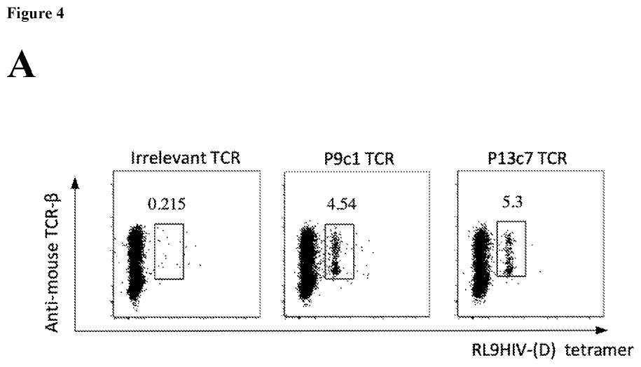

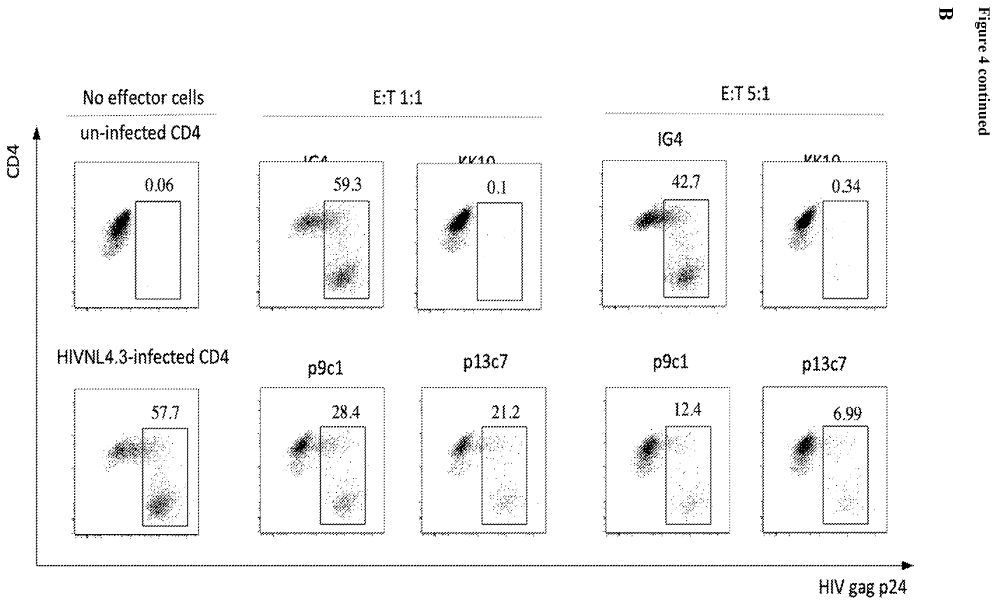

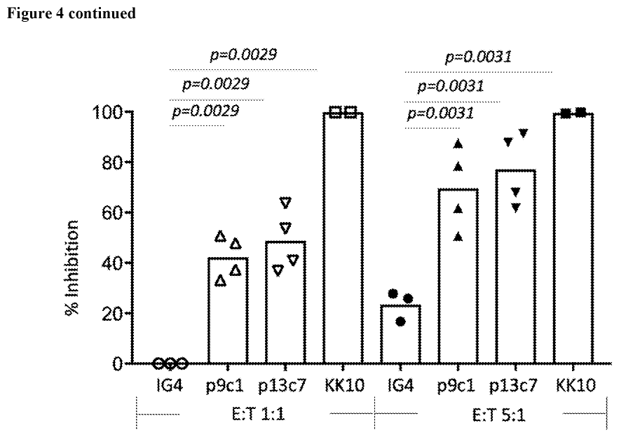

The αβ TCRs from the 14 clones. 12 of the 13 TCR sequences obtained were unique and one was present in two clones (Table 1). The TCR Va/Vβ from clones p9c1 and p13c7 was transduced, fused to murine Ca/Cβ, into primary CD8+ T cells. CD8+ T cell transductants were stained with disulphide trapped HLA-E-RL9 tetramers at day 4 post-transfection (FIG. 4A) and mouse CB-positive cells were sorted to enrich for RL9-specific TCR expressing cells. These were cultured in RPMI 1640 CM (10% AB serum) with IL-15 and IL-2 for a further 17 days before functional analyses. Then the CD8+ T cell TCR transductants were tested on activated primary CD4+ T cells from healthy allogeneic donors infected with HIV-1 NL4.3 virus, cultured at E: T ratios of 1:1 and 5:1 for 5 days (FIG. 4B). An irrelevant TCR specific for HLAA*0201-NY-ESO-1157-165 (SLLMWITQC) (IG4) was transduced into CD8 T cells from the same donor and included as a negative control, whilst an HLA B*2705 HIV-1 Gag263-272 (KRWIILGLNK) (KK10) specific TCR transduced CD8+ T cells was included as a positive control because the CD4+ T cells were purified from a B*2705+ donor. Both the p9c1 and p13c7 RL9-specific TCR CD8+ transductants significantly diminished HIV-1 virus replication, reducing the % p24+ cells by a mean of 42.3% and 48.8% respectively at an E: T ratio of 1:1 and by of 69.7% and 77.2%, at a ratio of 5:1. No reduction of p24+ cells was observed with the irrelevant TCR transduced cells at 1:1 and 23.5% at 5:1, whilst a 99.9% reduction was seen with KK10 TCR transduced cells at both ratios (FIG. 4B). However KK10 TCR transduced cells showed no inhibition on HIV-1 infected CD4 T cells that were HLA B*2705 negative.

The CD8+ T cell TCR transductants up-regulated CD137 expression and/or produced TNFα when stimulated with either RL9 peptide pulsed autologous EBV transformed B cells (FIG. 4C) or HIV-1 NL4.3-infected 721.221-CD4 cells (FIG. 4D), and responses were partially blocked by competitive inhibition with the HLA-E binding canonical signal peptide VL9, confirming presentation of RL9 by HLA-E.

### Example 5—HIV1 Suppresion by IL9 Specific, HLA-E Restricted TCRs.

Activated CD4+ T-cells were activated with HIVNL4.3 virus, and T-cell clones of interest added and co-cultured for 5-7 days. HIV replication was quantified; intracellular HIV1 gag was stained for. Results are shown below for each TCR clone.

| % suppression | TCR clone |
|---|---|
| 97 | Rev33 |
| 86 | Rev141 |
| 70.3 | Rev152 |
| 83.8 | Rev160 |
| 73.1 | Rev164 |
| 79.5 | Rev166 |
| 82.1 | Rev169 |
| 77.5 | Rev178 |
| 89 | Rev185 |
| 75.1 | Rev189 |
| 71.6 | Rev193 |
| 71.8 | Rev197 |
| 80.8 | Rev198 |
| 91.8 | Rev203 |
| 72.5 | Rev204 |
| 89.9 | Rev221 |
| 74.7 | Rev226 |
| 70 | Rev229 |
| 89.8 | Rev230 |

### Discussion

HLA-E restricted HIV specific CD8+ T cell responses have until now not been reported. In this study the best binding HIV-1 peptide, Gag 277-285 RMYSPTSIL (RL9) was taken and used to make a stable HLA-E tetramer which detected low frequencies of blood-derived CD8+ T cells in healthy blood donors. It is demonstrated that these T cells could be expanded by culture with peptide pulsed activated autologous dendritic cells and that RL9 specific T cell clones could then be generated. These T cells were polyclonal and capable of responding to cells presenting HLA-E-RL9 peptide. Critically these T cell clones could suppress HIV replication when cultured with HIV infected CD4+ cell lines. A similar approach was used to identify Rev 102-110 ILVESPAVL (IL9) specific T-cell clones.

HLA-E is intimately involved in natural killer cell recognition of target cells, however, expression of the receptor NKG2A/C-CD94 was not present on any of the T cell clones studied here. Additionally the validity of T cell responses was confirmed by transferring the T cell receptors of the clones, using lentiviral transduction, to CD8+ T cells from PBMC of an allogenic donor. In both types of transductant the TCR transferred the specificity for peptide pulsed and single chain trimer expressing targets and crucially, for HIV infected cells.

The recognition of HIV-1 infected cells by T cell clones and CD8 T cells transduced with the same TCRs demonstrates that human T cells can process the RL9 or IL9 peptide bound to HLA-E and present these molecules on their surface. Furthermore, it is clear that HIV-1 specific HLA-E restricted T cells can be generated from the PBMC of HIV seronegative blood donors. It is likely that these T cells are not elicited when there are strong classical MHC-Ia restricted T cell responses; if immunodominant, these could suppress or out-compete atypical responses. Alternatively, these cells might be present at very low frequencies, but responses to the RL9 peptide were not detected in any donors in the CHAVI001 acute HIV infection cohort. Similarly the RL9 peptide is not reported as a CD8+ T cell epitope in the LANL T-cell epitope database (https://www.hiv.lanl.gov/content/immunology/ctl_index.html). Here it is shown that HIV infected cells can be recognised by the T cell clones and TCR transductants because they present HLA-E-RL9 or HLA-E-IL9 at the cell surface. Mamu-E restricted T cells responding to the SIV homolog of RL9 in RhCMV68-1-SIV vaccinated monkeys also recognize and suppress SIV infected cells but SIV infected animals do not naturally make these T cell responses. One possible explanation is that there is a large quantitative difference in the amount of peptide-MHC needed for priming a T cell response compared to being recognized as a target. It is known that CD8+ T cell clones can recognise <10 molecules per cell. Although it has not yet been possible to accurately calculate how much peptide-MHC is need on a dendritic cell to prime naïve T cells, it is very likely to be orders of magnitude larger.

In conclusion, it is demonstrated that that human CD8+ T cells can respond to at least two HIV-1 peptides restricted by HLA-E and that the epitope is present on the surface of HIV-1 infected CD4+ T cells. Individual TCRs which can recognise HLA-E bound RL9 or IL9 and which can induce a CD8+ T-cell response to infected cells and reduce viral load has therefore been demonstrated for the first time.

1. A polypeptide encoding a T-cell receptor (TCR), wherein the TCR is virus specific and HLA-E restricted.

2. The polypeptide of claim 1, wherein the virus is HIV-1.

3. The polypeptide of claim 1 or claim 2, wherein the TCR is capable of binding to a peptide of RMYSPTSIL (SEQ ID NO: 1) or a peptide of SEQ ID NO: 1 in complex with HLA-E; or
wherein the TCR is capable of binding to a peptide of ILVESPAVL (SEQ ID NO: 110) or a peptide of SEQ ID NO: 110 in complex with HLA-E.

4. The polypeptide of any of claims 1-3, wherein the TCR comprises an alpha chain variable domain and a beta chain variable domain comprising:
a) a CDR3-alpha of CAFMKLHSGAGSYQLTF (SEQ ID NO: 2) or a CDR3-alpha with at least 90% identity, such as 90%, 95%, 96%, 97%, 98%, 99% of 100% identity to SEQ ID NO: 2, and a CDR3-beta of CASSLWAVGYGYTF (SEQ ID NO: 3) or a CDR3-beta with at least 90% identity, such as 90%, 95%, 96%, 97%, 98%, 99% of 100% identity to SEQ ID NO: 3; or
b) a CDR3-alpha of CAFDNNNDMRF (SEQ ID NO: 4) or a CDR3-alpha with at least 90% identity, such as 90%, 95%, 96%, 97%, 98%, 99% of 100% identity to SEQ ID NO: 4, and a CDR3-beta of CASSLVGAITEAFF (SEQ ID NO: 5) or a CDR3-beta with at least 90% identity, such as 90%, 95%, 96%, 97%, 98%, 99% of 100% identity to SEQ ID NO: 5; or
c) a CDR3-alpha of CAFVDGAGSYQLTF (SEQ ID NO: 6) or a CDR3-alpha with at least 90% identity, such as 90%, 95%, 96%, 97%, 98%, 99% of 100% identity to SEQ ID NO: 6, and a CDR3-beta of CASSVGNSNSPLHF (SEQ ID NO: 7) or a CDR3-beta with at least 90% identity, such as 90%, 95%, 96%, 97%, 98%, 99% of 100% identity to SEQ ID NO: 7; or
d) a CDR3-alpha of CAASGLFIQGGSEKLVF (SEQ ID NO: 8) or a CDR3-alpha with at least 90% identity, such as 90%, 95%, 96%, 97%, 98%, 99% of 100% identity to SEQ ID NO: 8, and a CDR3-beta of CASSVGNSNSPLHF (SEQ ID NO: 9) or a CDR3-beta with at least 90% identity, such as 90%, 95%, 96%, 97%, 98%, 99% of 100% identity to SEQ ID NO: 9; or
e) a CDR3-alpha of CAVYGSGKLTF (SEQ ID NO: 10) or a CDR3-alpha with at least 90% identity, such as 90%, 95%, 96%, 97%, 98%, 99% of 100% identity to SEQ ID NO: 10, and a CDR3-beta of CASSFGPSSGANVLTF (SEQ ID NO: 11) or a CDR3-beta with at least 90% identity, such as 90%, 95%, 96%, 97%, 98%, 99% of 100% identity to SEQ ID NO: 11; or
f) a CDR3-alpha of CAFTLYSGGGADGLTF (SEQ ID NO: 12) or a CDR3-alpha with at least 90% identity, such as 90%, 95%, 96%, 97%, 98%, 99% of 100% identity to SEQ ID NO: 12, and a CDR3-beta of CASSLPTSLSTDTQYF (SEQ ID NO: 13) or a CDR3-beta with at least 90% identity, such as 90%, 95%, 96%, 97%, 98%, 99% of 100% identity to SEQ ID NO: 13; or
g) a CDR3-alpha of CAMSWNSGNTPLVF (SEQ ID NO: 14) or a CDR3-alpha with at least 90% identity, such as 90%, 95%, 96%, 97%, 98%, 99% of 100% identity to SEQ ID NO: 14, and a CDR3-beta of CASSVTGVRNTIYF (SEQ ID NO: 15) or a CDR3-beta with at least 90% identity, such as 90%, 95%, 96%, 97%, 98%, 99% of 100% identity to SEQ ID NO: 15; or
h) a CDR3-alpha of CAAYGQKLLF (SEQ ID NO: 16) or a CDR3-alpha with at least 90% identity, such as 90%, 95%, 96%, 97%, 98%, 99% of 100% identity to SEQ ID NO: 16, and a CDR3-beta of CASSLLEPDLNTGELFF (SEQ ID NO: 17) or a CDR3-beta with at least 90% identity, such as 90%, 95%, 96%, 97%, 98%, 99% of 100% identity to SEQ ID NO: 17; or
i) a CDR3-alpha of CAVNTDKLIF (SEQ ID NO: 18) or a CDR3-alpha with at least 90% identity, such as 90%, 95%, 96%, 97%, 98%, 99% of 100% identity to SEQ ID NO: 18, and a CDR3-beta of CASSNPGNSDF (SEQ ID NO: 19) or a CDR3-beta with at least 90% identity, such as 90%, 95%, 96%, 97%, 98%, 99% of 100% identity to SEQ ID NO: 19; or
j) a CDR3-alpha of CAAVSTGSARQLTF (SEQ ID NO: 20) or a CDR3-alpha with at least 90% identity, such as 90%, 95%, 96%, 97%, 98%, 99% of 100% identity to SEQ ID NO: 20, and a CDR3-beta of CASSLAKGANVLTF (SEQ ID NO: 21) or a CDR3-beta with at least 90% identity, such as 90%, 95%, 96%, 97%, 98%, 99% of 100% identity to SEQ ID NO: 21; or
k) a CDR3-alpha of CVVSAWDPAAGNKLTF (SEQ ID NO: 22) or a CDR3-alpha with at least 90% identity, such as 90%, 95%, 96%, 97%, 98%, 99% of 100% identity to SEQ ID NO: 22, and a CDR3-beta of CASSPGGQGLDTQYF (SEQ ID NO: 23) or a CDR3-beta with at least 90% identity, such as 90%, 95%, 96%, 97%, 98%, 99% of 100% identity to SEQ ID NO: 23; or
l) a CDR3-alpha of CAYNRNDMRF (SEQ ID NO: 24) or a CDR3-alpha with at least 90% identity, such as 90%, 95%, 96%, 97%, 98%, 99% of 100% identity to SEQ ID NO: 24, and a CDR3-beta of CASSTDRDNQPQHF (SEQ ID NO: 25) or a CDR3-beta with at least 90% identity, such as 90%, 95%, 96%, 97%, 98%, 99% of 100% identity to SEQ ID NO: 25; or
m) a CDR3-alpha of CALSEALTSGTYKYIF (SEQ ID NO: 26) or a CDR3-alpha with at least 90% identity, such as 90%, 95%, 96%, 97%, 98%, 99% of 100% identity to SEQ ID NO: 26, and a CDR3-beta of CSASVGKSSYEQYV (SEQ ID NO: 27) or a CDR3-beta with at least 90% identity, such as 90%, 95%, 96%, 97%, 98%, 99% of 100% identity to SEQ ID NO: 27;or
n) a CDR3-alpha of CAVLLITQGGSEKLVF (SEQ ID NO: 111) or a CDR3-alpha with at least 90% identity, such as 90%, 95%, 96%, 97%, 98%, 99% of 100% identity to SEQ ID NO: 111, and a CDR3-beta of CASSVGGTNTQYF (SEQ ID NO: 112) or a CDR3-beta with at least 90% identity, such as 90%, 95%, 96%, 97%, 98%, 99% of 100% identity to SEQ ID NO: 112; or
o) a CDR3-alpha of CAVRDEDARLMF (SEQ ID NO: 113) or a CDR3-alpha with at least 90% identity, such as 90%, 95%, 96%, 97%, 98%, 99% of 100% identity to SEQ ID NO: 113, and a CDR3-beta of CSARGGGNRESHYGYTF (SEQ ID NO: 114) or a CDR3-beta with at least 90% identity, such as 90%, 95%, 96%, 97%, 98%, 99% of 100% identity to SEQ ID NO: 114; or
p) a CDR3-alpha of CAGQYSGGGADGLTF (SEQ ID NO: 115) or a CDR3-alpha with at least 90% identity, such as 90%, 95%, 96%, 97%, 98%, 99% of 100% identity to SEQ ID NO: 115, and a CDR3-beta of CASSLPDSSETQYF (SEQ ID NO: 116) or a CDR3-beta with at least 90% identity, such as 90%, 95%, 96%, 97%, 98%, 99% of 100% identity to SEQ ID NO: 116; or
q) a CDR3-alpha of CALSEYGGATNKLIF (SEQ ID NO: 117) or a CDR3-alpha with at least 90% identity, such as 90%, 95%, 96%, 97%, 98%, 99% of 100% identity to SEQ ID NO: 117, and a CDR3-beta of CATSRFLEGKDTEAFF (SEQ ID NO: 118) or a CDR3-beta with at least 90% identity, such as 90%, 95%, 96%, 97%, 98%, 99% of 100% identity to SEQ ID NO: 118; or
r) a CDR3-alpha of CAGGGGADGLTF (SEQ ID NO: 119) or a CDR3-alpha with at least 90% identity, such as 90%, 95%, 96%, 97%, 98%, 99% of 100% identity to SEQ ID NO: 119, and a CDR3-beta of CSVAETGTEAFF (SEQ ID NO: 120) or a CDR3-beta with at least 90% identity, such as 90%, 95%, 96%, 97%, 98%, 99% of 100% identity to SEQ ID NO: 120; or
s) a CDR3-alpha of CALSEAYSGAGSYQLTF (SEQ ID NO: 121) or a CDR3-alpha with at least 90% identity, such as 90%, 95%, 96%, 97%, 98%, 99% of 100% identity to SEQ ID NO: 121, and a CDR3-beta of CASNVGEGYNQPQHF (SEQ ID NO: 122) or a CDR3-beta with at least 90% identity, such as 90%, 95%, 96%, 97%, 98%, 99% of 100% identity to SEQ ID NO: 122; or
t) a CDR3-alpha of CAAWAPTNFGNEKLTF (SEQ ID NO: 123) or a CDR3-alpha with at least 90% identity, such as 90%, 95%, 96%, 97%, 98%, 99% of 100% identity to SEQ ID NO: 123, and a CDR3-beta of CASSVGYPGELFF (SEQ ID NO: 124) or a CDR3-beta with at least 90% identity, such as 90%, 95%, 96%, 97%, 98%, 99% of 100% identity to SEQ ID NO: 124; or
u) a CDR3-alpha of CAETLTHGGATNKLIF (SEQ ID NO: 125) or a CDR3-alpha with at least 90% identity, such as 90%, 95%, 96%, 97%, 98%, 99% of 100% identity to SEQ ID NO: 125, and a CDR3-beta of CASSEPGAAYEQYF (SEQ ID NO: 126) or a CDR3-beta with at least 90% identity, such as 90%, 95%, 96%, 97%, 98%, 99% of 100% identity to SEQ ID NO: 126; or
v) a CDR3-alpha of CKGGAQKLVF (SEQ ID NO: 127) or a CDR3-alpha with at least 90% identity, such as 90%, 95%, 96%, 97%, 98%, 99% of 100% identity to SEQ ID NO: 127, and a CDR3-beta of CASSLGRSYEQYF (SEQ ID NO: 128) or a CDR3-beta with at least 90% identity, such as 90%, 95%, 96%, 97%, 98%, 99% of 100% identity to SEQ ID NO: 128; or
w) a CDR3-alpha of CAVRDRNNFNKFYF (SEQ ID NO: 129) or a CDR3-alpha with at least 90% identity, such as 90%, 95%, 96%, 97%, 98%, 99% of 100% identity to SEQ ID NO: 129, and a CDR3-beta of CASSGVKGTGSEKLFF (SEQ ID NO: 130) or a CDR3-beta with at least 90% identity, such as 90%, 95%, 96%, 97%, 98%, 99% of 100% identity to SEQ ID NO: 130; or
x) a CDR3-alpha of CAMREGAGAGSYQLTF (SEQ ID NO: 131) or a CDR3-alpha with at least 90% identity, such as 90%, 95%, 96%, 97%, 98%, 99% of 100% identity to SEQ ID NO: 131, and a CDR3-beta of CASLAGQGRSEAFF (SEQ ID NO: 132) or a CDR3-beta with at least 90% identity, such as 90%, 95%, 96%, 97%, 98%, 99% of 100% identity to SEQ ID NO: 132; or
y) a CDR3-alpha of CAVSTGGGNKLTF (SEQ ID NO: 133) or a CDR3-alpha with at least 90% identity, such as 90%, 95%, 96%, 97%, 98%, 99% of 100% identity to SEQ ID NO: 133, and a CDR3-beta of CASSDGLRGSVRYEQYF (SEQ ID NO: 134) or a CDR3-beta with at least 90% identity, such as 90%, 95%, 96%, 97%, 98%, 99% of 100% identity to SEQ ID NO: 134; or
z) a CDR3-alpha of CAVVWATRLMF (SEQ ID NO: 135) or a CDR3-alpha with at least 90% identity, such as 90%, 95%, 96%, 97%, 98%, 99% of 100% identity to SEQ ID NO: 135, and a CDR3-beta of CAIGGQEILMHGYTF (SEQ ID NO: 136) or a CDR3-beta with at least 90% identity, such as 90%, 95%, 96%, 97%, 98%, 99% of 100% identity to SEQ ID NO: 136; or
aa) a CDR3-alpha of CAATFVSGSARQLTF (SEQ ID NO: 137) or a CDR3-alpha with at least 90% identity, such as 90%, 95%, 96%, 97%, 98%, 99% of 100% identity to SEQ ID NO: 137, and a CDR3-beta of CASSLRRAHTDTQYF (SEQ ID NO: 138) or a CDR3-beta with at least 90% identity, such as 90%, 95%, 96%, 97%, 98%, 99% of 100% identity to SEQ ID NO: 138; or
bb) a CDR3-alpha of CALSRRYSTLTF (SEQ ID NO: 139) or a CDR3-alpha with at least 90% identity, such as 90%, 95%, 96%, 97%, 98%, 99% of 100% identity to SEQ ID NO: 139, and a CDR3-beta of CASRLTDSYEQYF (SEQ ID NO: 140) or a CDR3-beta with at least 90% identity, such as 90%, 95%, 96%, 97%, 98%, 99% of 100% identity to SEQ ID NO: 140; or
cc) a CDR3-alpha of CAAGSSNTGKLIF (SEQ ID NO: 141) or a CDR3-alpha with at least 90% identity, such as 90%, 95%, 96%, 97%, 98%, 99% of 100% identity to SEQ ID NO: 141, and a CDR3-beta of CSVEFASKGYEQYF (SEQ ID NO: 142) or a CDR3-beta with at least 90% identity, such as 90%, 95%, 96%, 97%, 98%, 99% of 100% identity to SEQ ID NO: 142; or
dd) a CDR3-alpha of CAATPLLGADKIIF (SEQ ID NO: 143) or a CDR3-alpha with at least 90% identity, such as 90%, 95%, 96%, 97%, 98%, 99% of 100% identity to SEQ ID NO: 143, and a CDR3-beta of CASSFRGEAEAFF (SEQ ID NO: 144) or a CDR3-beta with at least 90% identity, such as 90%, 95%, 96%, 97%, 98%, 99% of 100% identity to SEQ ID NO: 144; or
ee) a CDR3-alpha of CAGSGYSGAGSYQLTF (SEQ ID NO: 145) or a CDR3-alpha with at least 90% identity, such as 90%, 95%, 96%, 97%, 98%, 99% of 100% identity to SEQ ID NO: 145, and a CDR3-beta of CASSFSAGTDTQYF (SEQ ID NO: 146) or a CDR3-beta with at least 90% identity, such as 90%, 95%, 96%, 97%, 98%, 99% of 100% identity to SEQ ID NO: 146; or
ff) a CDR3-alpha of CAVSSQVTGGSEKLVF (SEQ ID NO: 147) or a CDR3-alpha with at least 90% identity, such as 90%, 95%, 96%, 97%, 98%, 99% of 100% identity to SEQ ID NO: 147, and a CDR3-beta of CASSPPTLGYGYTF (SEQ ID NO: 148) or a CDR3-beta with at least 90% identity, such as 90%, 95%, 96%, 97%, 98%, 99% of 100% identity to SEQ ID NO: 148.

5. The polypeptide of any of claims 1-3, wherein the TCR comprises:
a) an alpha chain variable domain comprising a CDR1-alpha of TSENNY (SEQ ID NO: 28), a CDR2-alpha of QEAYKQQN (SEQ ID NO: 29) and a CDR3-alpha of SEQ ID NO: 2; and/or a beta alpha chain variable domain comprising a CDR1-beta of SGHDY (SEQ ID NO: 30), a CDR2-beta of FNNNVP (SEQ ID NO: 31) and a CDR3-beta of SEQ ID NO: 3; or
b) an alpha chain variable domain comprising a CDR1-alpha of TSENNY (SEQ ID NO: 32), a CDR2-alpha of QEAYKQQN (SEQ ID NO: 33) and a CDR3-alpha of SEQ ID NO: 4; and/or a beta alpha chain variable domain comprising a CDR1-beta of SGHDT (SEQ ID NO: 34), a CDR2-beta of YYEEEE (SEQ ID NO: 35) and a CDR3-beta of SEQ ID NO: 5; or
c) an alpha chain variable domain comprising a CDR1-alpha of TSENNY (SEQ ID NO: 36), a CDR2-alpha of QEAYKQQN (SEQ ID NO: 37) and a CDR3-alpha of SEQ ID NO: 6; and/or a beta alpha chain variable domain comprising a CDR1-beta of SGDLS (SEQ ID NO: 38), a CDR2-beta of YYNGEE (SEQ ID NO: 39) and a CDR3-beta of SEQ ID NO:7; or
d) an alpha chain variable domain comprising a CDR1-alpha of NTAFDY (SEQ ID NO: 40), a CDR2-alpha of IRPDVSE (SEQ ID NO: 41) and a CDR3-alpha of SEQ ID NO: 8; and/or a beta alpha chain variable domain comprising a CDR1-beta of SGDLS (SEQ ID NO: 42), a CDR2-beta of YYNGEE (SEQ ID NO: 43) and a CDR3-beta of SEQ ID NO: 9; or
e) an alpha chain variable domain comprising a CDR1-alpha of DRGSQS (SEQ ID NO: 44), a CDR2-alpha of IYSNGD (SEQ ID NO: 45) and a CDR3-alpha of SEQ ID NO: 10; and/or a beta alpha chain variable domain comprising a CDR1-beta of MDHEN (SEQ ID NO: 46), a CDR2-beta of SYDVKM (SEQ ID NO: 47) and a CDR3-beta of SEQ ID NO: 11; or
f) an alpha chain variable domain comprising a CDR1-alpha of TSENNYY (SEQ ID NO: 48), a CDR2-alpha of QEAYKQQN (SEQ ID NO: 49) and a CDR3-alpha of SEQ ID NO: 12; and/or a beta alpha chain variable domain comprising a CDR1-beta of MDHEN (SEQ ID NO: 50), a CDR2-beta of SYDVKM (SEQ ID NO: 51) and a CDR3-beta of SEQ ID NO: 13; or
g) an alpha chain variable domain comprising a CDR1-alpha of NSAFQY (SEQ ID NO: 52), a CDR2-alpha of TYSSGN (SEQ ID NO: 53) and a CDR3-alpha of SEQ ID NO: 14; and/or a beta alpha chain variable domain comprising a CDR1-beta of SNHLY (SEQ ID NO: 54), a CDR2-beta of FYNNEI (SEQ ID NO: 55) and a CDR3-beta of SEQ ID NO: 15; or
h) The TCR may comprise an alpha chain variable domain comprising a CDR1-alpha of DSASNY (SEQ ID NO: 56), a CDR2-alpha of IRSNVGE (SEQ ID NO: 57) and a CDR3-alpha of SEQ ID NO: 16; and/or a beta alpha chain variable domain comprising a CDR1-beta of MDHEN (SEQ ID NO: 58), a CDR2-beta of SYDVKM (SEQ ID NO: 59) and a CDR3-beta of SEQ ID NO: 17; or
i) an alpha chain variable domain comprising a CDR1-alpha of TSGFNG (SEQ ID NO: 60), a CDR2-alpha of NVLDGL (SEQ ID NO: 61) and a CDR3-alpha of SEQ ID NO: 18; and/or a beta alpha chain variable domain comprising a CDR1-beta of SEHNR (SEQ ID NO: 62), a CDR2-beta of FQNEAQ (SEQ ID NO: 63) and a CDR3-beta of SEQ ID NO: 19; or
j) an alpha chain variable domain comprising a CDR1-alpha of NSMFDY (SEQ ID NO: 64), a CDR2-alpha of ISSIKDK (SEQ ID NO: 65) and a CDR3-alpha of SEQ ID NO: 20; and/or a beta alpha chain variable domain comprising a CDR1-beta of SGHRS (SEQ ID NO: 66), a CDR2-beta of YFSETQ (SEQ ID NO: 67) and a CDR3-beta of SEQ ID NO: 21; or
k) an alpha chain variable domain comprising a CDR1-alpha of VSPFSN (SEQ ID NO: 68), a CDR2-alpha of MTFSENT (SEQ ID NO: 69) and a CDR3-alpha of SEQ ID NO: 22; and/or a beta alpha chain variable domain comprising a CDR1-beta of MNHNY (SEQ ID NO: 70), a CDR2-beta of SVGAGI (SEQ ID NO: 71) and a CDR3-beta of SEQ ID NO: 23; or
l) an alpha chain variable domain comprising a CDR1-alpha of TSESDYY (SEQ ID NO: 72), a CDR2-alpha of QEAYKQQN (SEQ ID NO: 73) and a CDR3-alpha of SEQ ID NO: 24; and/or a beta alpha chain variable domain comprising a CDR1-beta of LNHDA (SEQ ID NO: 74), a CDR2-beta of SQIVND (SEQ ID NO: 75) and a CDR3-beta of SEQ ID NO: 25; or
m) an alpha chain variable domain comprising a CDR1-alpha of TRDTTYY (SEQ ID NO: 76), a CDR2-alpha of RNSFDEQN (SEQ ID NO: 77) and a CDR3-alpha of SEQ ID NO: 26; and/or a beta alpha chain variable domain comprising a CDR1-beta of DFQATT (SEQ ID NO: 78), a CDR2-beta of SNEGSKA (SEQ ID NO: 79) and a CDR3-beta of SEQ ID NO: 27.

6. The polypeptide of any of any of claims 1-3, wherein the TCR comprises:
a) an alpha chain variable domain comprising a variable segment encoded by the TRAV38-1 gene, a Joining segment encoded by the TRAJ28 gene, a CDR1-alpha of SEQ ID NO: 28, a CDR2-alpha of SEQ ID NO: 29 and a CDR3-alpha of SEQ ID NO: 2; and/or a beta chain variable region comprising a variable segment encoded by the TRBV12-4 gene, a Joining segment encoded by the TRBJ1-2 gene, a CDR1-beta of SEQ ID NO: 30, a CDR2-beta of SEQ ID NO: 31 and a CDR3-beta of SEQ ID NO: 3; or
b) an alpha chain variable domain comprising a variable segment encoded by the TRAV38-1 gene, a Joining segment encoded by the TRAJ43 gene, a CDR1-alpha of SEQ ID NO: 32, a CDR2-alpha of SEQ ID NO: 33 and a CDR3-alpha of SEQ ID NO: 4; and/or a beta chain variable region comprising a variable segment encoded by the TRBV5-6 gene, a Joining segment encoded by the TRBJ1-1 gene, a CDR1-beta of SEQ ID NO: 34, a CDR2-beta of SEQ ID NO: 35 and a CDR3-beta of SEQ ID NO: 5; or
c) an alpha chain variable domain comprising a variable segment encoded by the TRAV38-1 gene, a Joining segment encoded by the TRAJ28 gene, a CDR1-alpha of SEQ ID NO: 36, a CDR2-alpha of SEQ ID NO: 37 and a CDR3-alpha of SEQ ID NO: 6; and/or a beta chain variable region comprising a variable segment encoded by the TRBV9 gene, a Joining segment encoded by the TRBJ1-6 gene, a CDR1-beta of SEQ ID NO: 38, a CDR2-beta of SEQ ID NO: 39 and a CDR3-beta of SEQ ID NO: 7; or
d) comprise an alpha chain variable domain comprising a variable segment encoded by the TRAV23DV6 gene, a Joining segment encoded by the TRAJ57 gene, a CDR1-alpha of SEQ ID NO: 40, a CDR2-alpha of SEQ ID NO: 41 and a CDR3-alpha of SEQ ID NO: 8; and/or a beta chain variable region comprising a variable segment encoded by the TRBV9 gene, a Joining segment encoded by the TRBJ1-6 gene, a CDR1-beta of SEQ ID NO: 42, a CDR2-beta of SEQ ID NO: 43 and a CDR3-beta of SEQ ID NO: 9; or
e) an alpha chain variable domain comprising a variable segment encoded by the TRAV12-2 gene, a Joining segment encoded by the TRAJ48 gene, a CDR1-alpha of SEQ ID NO: 44, a CDR2-alpha of SEQ ID NO: 45 and a CDR3-alpha of SEQ ID NO: 10; and/or a beta chain variable region comprising a variable segment encoded by the TRBV28 gene, a Joining segment encoded by the TRBJ2-6 gene, a CDR1-beta of SEQ ID NO: 46, a CDR2-beta of SEQ ID NO: 47 and a CDR3-beta of SEQ ID NO: 11; or
f) an alpha chain variable domain comprising a variable segment encoded by the TRAV38-1 gene, a Joining segment encoded by the TRAJ45 gene, a CDR1-alpha of SEQ ID NO: 48, a CDR2-alpha of SEQ ID NO: 49 and a CDR3-alpha of SEQ ID NO: 12; and/or a beta chain variable region comprising a variable segment encoded by the TRBV28 gene, a Joining segment encoded by the TRBJ2-3 gene, a CDR1-beta of SEQ ID NO: 50, a CDR2-beta of SEQ ID NO: 51 and a CDR3-beta of SEQ ID NO: 13; or
g) comprise an alpha chain variable domain comprising a variable segment encoded by the TRAV12-3 gene, a Joining segment encoded by the TRAJ29 gene, a CDR1-alpha of SEQ ID NO: 52, a CDR2-alpha of SEQ ID NO: 53 and a CDR3-alpha of SEQ ID NO: 14; and/or a beta chain variable region comprising a variable segment encoded by the TRBV2 gene, a Joining segment encoded by the TRBJ1-3 gene, a CDR1-beta of SEQ ID NO: 54, a CDR2-beta of SEQ ID NO: 55 and a CDR3-beta of SEQ ID NO: 15; or
h) an alpha chain variable domain comprising a variable segment encoded by the TRAV13-1 gene, a Joining segment encoded by the TRAJ16 gene, a CDR1-alpha of SEQ ID NO: 56, a CDR2-alpha of SEQ ID NO: 57 and a CDR3-alpha of SEQ ID NO: 16; and/or a beta chain variable region comprising a variable segment encoded by the TRBV28 gene, a Joining segment encoded by the TRBJ2-2 gene, a CDR1-beta of SEQ ID NO: 58, a CDR2-beta of SEQ ID NO: 59 and a CDR3-beta of SEQ ID NO: 17; or
i) an alpha chain variable domain comprising a variable segment encoded by the TRAV1-2 gene, a Joining segment encoded by the TRAJ34 gene, a CDR1-alpha of SEQ ID NO: 60, a CDR2-alpha of SEQ ID NO: 61 and a CDR3-alpha of SEQ ID NO: 18; and/or a beta chain variable region comprising a variable segment encoded by the TRBV7-9 gene, a Joining segment encoded by the TRBJ2-3 gene, a CDR1-beta of SEQ ID NO: 62, a CDR2-beta of SEQ ID NO: 63 and a CDR3-beta of SEQ ID NO: 19; or
j) an alpha chain variable domain comprising a variable segment encoded by the TRAV29DV5 gene, a Joining segment encoded by the TRAJ22 gene, a CDR1-alpha of SEQ ID NO: 64, a CDR2-alpha of SEQ ID NO: 65 and a CDR3-alpha of SEQ ID NO: 20; and/or a beta chain variable region comprising a variable segment encoded by the TRBVS-1 gene, a Joining segment encoded by the TRBJ2-6 gene, a CDR1-beta of SEQ ID NO: 66, a CDR2-beta of SEQ ID NO: 67 and a CDR3-beta of SEQ ID NO: 21; or
k) an alpha chain variable domain comprising a variable segment encoded by the TRAV10 gene, a Joining segment encoded by the TRAJ17 gene, a CDR1-alpha of SEQ ID NO: 68, a CDR2-alpha of SEQ ID NO: 69 and a CDR3-alpha of SEQ ID NO: 22; and/or a beta chain variable region comprising a variable segment encoded by the TRBV6-6 gene, a Joining segment encoded by the TRBJ2-3 gene, a CDR1-beta of SEQ ID NO: 70, a CDR2-beta of SEQ ID NO: 71 and a CDR3-beta of SEQ ID NO: 23; or
l) an alpha chain variable domain comprising a variable segment encoded by the TRAV38-2DV8 gene, a Joining segment encoded by the TRAJ43 gene, a CDR1-alpha of SEQ ID NO: 72, a CDR2-alpha of SEQ ID NO: 73 and a CDR3-alpha of SEQ ID NO: 24; and/or a beta chain variable region comprising a variable segment encoded by the TRBV19 gene, a Joining segment encoded by the TRBJ1-5 gene, a CDR1-beta of SEQ ID NO: 74, a CDR2-beta of SEQ ID NO: 75 and a CDR3-beta of SEQ ID NO: 25; or
m) an alpha chain variable domain comprising a variable segment encoded by the TRAV19 gene, a Joining segment encoded by the TRAJ40 gene, a CDR1-alpha of SEQ ID NO: 76, a CDR2-alpha of SEQ ID NO: 77 and a CDR3-alpha of SEQ ID NO: 26; and/or a beta chain variable region comprising a variable segment encoded by the TRBV20 gene, a Joining segment encoded by the TRBJ2-7 gene, a CDR1-beta of SEQ ID NO: 78, a CDR2-beta of SEQ ID NO: 79 and a CDR3-beta of SEQ ID NO: 27 or;
n) an alpha chain variable domain comprising a variable segment encoded by the TRAV41 gene, a Joining segment encoded by the TRAJ57 gene, a CDR3-alpha of SEQ ID NO: 111; and/or a beta chain variable region comprising a variable segment encoded by the TRBV9 gene, a Joining segment encoded by the TRBJ2-3 gene, and a CDR3-beta of SEQ ID NO: 112; or
o) an alpha chain variable domain comprising a variable segment encoded by the TRAV1-2 gene, a Joining segment encoded by the TRAJ31 gene, a CDR3-alpha of SEQ ID NO: 113; and/or a beta chain variable region comprising a variable segment encoded by the TRBV20-1 gene, a Joining segment encoded by the TRBJ1-2 gene, and a CDR3-beta of SEQ ID NO: 114; or
p) an alpha chain variable domain comprising a variable segment encoded by the TRAV35 gene, a Joining segment encoded by the TRAJ45 gene, a CDR3-alpha of SEQ ID NO: 115; and/or a beta chain variable region comprising a variable segment encoded by the TRBVS-6 gene, a Joining segment encoded by the TRBJ2-5 gene, and a CDR3-beta of SEQ ID NO: 116; or
q) an alpha chain variable domain comprising a variable segment encoded by the TRAV19 gene, a Joining segment encoded by the TRAJ32 gene, a CDR3-alpha of SEQ ID NO: 117; and/or a beta chain variable region comprising a variable segment encoded by the TRBV15 gene, a Joining segment encoded by the TRBJ1-1 gene, and a CDR3-beta of SEQ ID NO: 118; or
r) an alpha chain variable domain comprising a variable segment encoded by the TRAV41 gene, a Joining segment encoded by the TRAJ45 gene, a CDR3-alpha of SEQ ID NO: 119; and/or a beta chain variable region comprising a variable segment encoded by the TRBV29-1 gene, a Joining segment encoded by the TRBJ1-1 gene, and a CDR3-beta of SEQ ID NO: 120; or
s) an alpha chain variable domain comprising a variable segment encoded by the TRAV19 gene, a Joining segment encoded by the TRAJ28 gene, a CDR3-alpha of SEQ ID NO: 121; and/or a beta chain variable region comprising a variable segment encoded by the TRBV19 gene, a Joining segment encoded by the TRBJ1-5 gene, and a CDR3-beta of SEQ ID NO: 122; or
t) an alpha chain variable domain comprising a variable segment encoded by the TRAV41 gene, a Joining segment encoded by the TRAJ48 gene, a CDR3-alpha of SEQ ID NO: 123; and/or a beta chain variable region comprising a variable segment encoded by the TRBV9 gene, a Joining segment encoded by the TRBJ2-2 gene, and a CDR3-beta of SEQ ID NO: 124; or
u) an alpha chain variable domain comprising a variable segment encoded by the TRAV13-2 gene, a Joining segment encoded by the TRAJ32 gene, a CDR3-alpha of SEQ ID NO: 125; and/or a beta chain variable region comprising a variable segment encoded by the TRBV2 gene, a Joining segment encoded by the TRBJ2-7 gene, and a CDR3-beta of SEQ ID NO: 126; or
v) an alpha chain variable domain comprising a variable segment encoded by the TRAV25 gene, a Joining segment encoded by the TRAJ54 gene, a CDR3-alpha of SEQ ID NO: 127; and/or a beta chain variable region comprising a variable segment encoded by the TRBV27 gene, a Joining segment encoded by the TRBJ2-7 gene, and a CDR3-beta of SEQ ID NO: 128; or
w) an alpha chain variable domain comprising a variable segment encoded by the TRAV3 gene, a Joining segment encoded by the TRAJ21 gene, a CDR3-alpha of SEQ ID NO: 129; and/or a beta chain variable region comprising a variable segment encoded by the TRBV28 gene, a Joining segment encoded by the TRBJ1-4 gene, and a CDR3-beta of SEQ ID NO: 130; or
x) an alpha chain variable domain comprising a variable segment encoded by the TRAV14DV4 gene, a Joining segment encoded by the TRAJ28 gene, a CDR3-alpha of SEQ ID NO: 131; and/or a beta chain variable region comprising a variable segment encoded by the TRBV9 gene, a Joining segment encoded by the TRBJ1-1 gene, and a CDR3-beta of SEQ ID NO: 132; or
y) an alpha chain variable domain comprising a variable segment encoded by the TRAV12-2 gene, a Joining segment encoded by the TRAJ10 gene, a CDR3-alpha of SEQ ID NO: 133; and/or a beta chain variable region comprising a variable segment encoded by the TRBV2 gene, a Joining segment encoded by the TRBJ2-7 gene, and a CDR3-beta of SEQ ID NO: 134; or
z) an alpha chain variable domain comprising a variable segment encoded by the TRAV39 gene, a Joining segment encoded by the TRAJ31 gene, a CDR3-alpha of SEQ ID NO: 135; and/or a beta chain variable region comprising a variable segment encoded by the TRBV10-3 gene, a Joining segment encoded by the TRBJ1-2 gene, and a CDR3-beta of SEQ ID NO: 136; or
aa) an alpha chain variable domain comprising a variable segment encoded by the TRAV29DV5 gene, a Joining segment encoded by the TRAJ22 gene, a CDR3-alpha of SEQ ID NO: 137; and/or a beta chain variable region comprising a variable segment encoded by the TRBV9 gene, a Joining segment encoded by the TRBJ2-3 gene, and a CDR3-beta of SEQ ID NO: 138; or
bb) an alpha chain variable domain comprising a variable segment encoded by the TRAV19 gene, a Joining segment encoded by the TRAJ11 gene, a CDR3-alpha of SEQ ID NO: 139; and/or a beta chain variable region comprising a variable segment encoded by the TRBV6-6 gene, a Joining segment encoded by the TRBJ2-7 gene, and a CDR3-beta of SEQ ID NO: 140; or
cc) an alpha chain variable domain comprising a variable segment encoded by the TRAV21 gene, a Joining segment encoded by the TRAJ37 gene, a CDR3-alpha of SEQ ID NO: 141; and/or a beta chain variable region comprising a variable segment encoded by the TRBV29-1 gene, a Joining segment encoded by the TRBJ2-7 gene, and a CDR3-beta of SEQ ID NO: 142; or
dd) an alpha chain variable domain comprising a variable segment encoded by the TRAV23DV6 gene, a Joining segment encoded by the TRAJ30 gene, a CDR3-alpha of SEQ ID NO: 143; and/or a beta chain variable region comprising a variable segment encoded by the TRBV27 gene, a Joining segment encoded by the TRBJ1-1 gene, and a CDR3-beta of SEQ ID NO: 144; or
ee) an alpha chain variable domain comprising a variable segment encoded by the TRAV21 gene, a Joining segment encoded by the TRAJ28 gene, a CDR3-alpha of SEQ ID NO: 145; and/or a beta chain variable region comprising a variable segment encoded by the TRBV27 gene, a Joining segment encoded by the TRBJ2-3 gene, and a CDR3-beta of SEQ ID NO: 146; or
ff) an alpha chain variable domain comprising a variable segment encoded by the TRAV21 gene, a Joining segment encoded by the TRAJ57 gene, a CDR3-alpha of SEQ ID NO: 147; and/or a beta chain variable region comprising a variable segment encoded by the TRBV28 gene, a Joining segment encoded by the TRBJ1-2 gene, and a CDR3-beta of SEQ ID NO: 148.

7. The polypeptide of any of claims 1-3, wherein the TCR comprises:
a) an alpha chain variable domain of a sequence of MTRVSLLWAVVVSTCLESGMAQTVTQSQPEMSVQEAETVTLSCTYDTSENNYY LFWYKQPPSRQMILVIRQEAYKQQNATENRFSVNFQKAAKSFSLKISDSQLGDTA MYFCAFMKLHSGAGSYQLTFGKGTKLSVIP (SEQ ID NO: 80) or a sequence with at least 90% identity to SEQ ID NO: 80, and a beta chain variable domain of a sequence of MGSWTLCCVSLCILVAKHTDAGVIQSPRHEVTEMGQEVTLRCKPISGHDYLFWY RQTMMRGLELLIYFNNNVPIDDSGMPEDRFSAKMPNASFSTLKIQPSEPRDSAVY FCASSLWAVGYGYTFGSGTRLTVV (SEQ ID NO: 81) or a sequence with at least 90% identity to SEQ ID NO: 81; or
b) an alpha chain variable domain of a sequence of MTRVSLLWAVVVSTCLESGMAQTVTQSQPEMSVQEAETVTLSCTYDTSENNYY LFWYKQPPSRQMILVIRQEAYKQQNATENRFSVNFQKAAKSFSLKISDSQLGDTA MYFCAFDNNNDMRFGAGTRLTVKP (SEQ ID NO: 82) or a sequence with at least 90% identity to SEQ ID NO: 82, and a beta chain variable domain of a sequence of MGPGLLCWALLCLLGAGLVDAGVTQSPTHLIKTRGQQVTLRCSPKSGHDTVSW YQQALGQGPQFIFQYYEEEERQRGNFPDRFSGHQFPNYSSELNVNALLLGDSALY LCASSLVGAITEAFFGQGTRLTVV (SEQ ID NO: 83) or a sequence with at least 90% identity to SEQ ID NO: 83; or
c) an alpha chain variable domain of a sequence of MTRVSLLWAVVVSTCLESGMAQTVTQSQPEMSVQEAETVTLSCTYDTSENNYY LFWYKQPPSRQMILVIRQEAYKQQNATENRFSVNFQKAAKSFSLKISDSQLGDTA MYFCAFVDGAGSYQLTFGKGTKLSVIP (SEQ ID NO: 84) or a sequence with at least 90% identity to SEQ ID NO: 84, and a beta chain variable domain of a sequence of MGFRLLCCVAFCLLGAGPVDSGVTQTPKHLITATGQRVTLRCSPRSGDLSVYWY QQSLDQGLQFLIQYYNGEERAKGNILERFSAQQFPDLHSELNLSSLELGDSALYFC ASSVGNSNSPLHFGNGTRLTVT (SEQ ID NO: 85) or a sequence with at least 90% identity to SEQ ID NO: 85; or
d) an alpha chain variable domain of a sequence of MDKILGASFLVLWLQLCWVSGQQKEKSDQQQVKQSPQSLIVQKGGISIINCAYEN TAFDYFPWYQQFPGKGPALLIAIRPDVSEKKEGRFTISFNKSAKQFSLHIMDSQPG DSATYFCAASGLFIQGGSEKLVFGKGMKLTVNP (SEQ ID NO: 86) or a sequence with at least 90% identity to SEQ ID NO: 86, and a beta chain variable domain of a sequence of MGFRLLCCVAFCLLGAGPVDSGVTQTPKHLITATGQRVTLRCSPRSGDLSVYWY QQSLDQGLQFLIQYYNGEERAKGNILERFSAQQFPDLHSELNLSSLELGDSALYFC ASSVGNSNSPLHFGNGTRLTVT (SEQ ID NO: 87) or a sequence with at least 90% identity to SEQ ID NO: 87; or
e) an alpha chain variable domain of a sequence of MKSLRVLLVILWLQLSWVWSQQKEVEQNSGPLSVPEGAIASLNCTYSDRGSQSFF WYRQYSGKSPELIMFIYSNGDKEDGRFTAQLNKASQYVSLLIRDSQPSDSATYLC AVYGSGKLTFGTGTRLTIIP (SEQ ID NO: 88) or a sequence with at least 90% identity to SEQ ID NO: 88, and a beta chain variable domain of a sequence of MGIRLLCRVAFCFLAVGLVDVKVTQSSRYLVKRTGEKVFLECVQDMDHENMFW YRQDPGLGLRLIYFSYDVKMKEKGDIPEGYSVSREKKERFSLILESASTNQTSMY LCASSFGPSSGANVLTFGAGSRLTVL (SEQ ID NO: 89) or a sequence with at least 90% identity to SEQ ID NO: 89; or
f) an alpha chain variable domain of a sequence of MTRVSLLWAVVVSTCLESGMAQTVTQSQPEMSVQEAETVTLSCTYDTSENNYY LFWYKQPPSRQMILVIRQEAYKQQNATENRFSVNFQKAAKSFSLKISDSQLGDTA MYFCAFTLYSGGGADGLTFGKGTHLIIQP (SEQ ID NO: 90) or a sequence with at least 90% identity to SEQ ID NO: 90, and a beta chain variable domain of a sequence of MGIRLLCRVAFCFLAVGLVDVKVTQSSRYLVKRTGEKVFLECVQDMDHENMFW YRQDPGLGLRLIYFSYDVKMKEKGDIPEGYSVSREKKERFSLILESASTNQTSMY LCASSLPTSLSTDTQYFGPGTRLTVL (SEQ ID NO: 91) or a sequence with at least 90% identity to SEQ ID NO: 91; or
g) an alpha chain variable domain of a sequence of MMKSLRVLLVILWLQLSWVWSQQKEVEQDPGPLSVPEGAIVSLNCTYSNSAFQY FMWYRQYSRKGPELLMYTYSSGNKEDGRFTAQVDKSSKYISLFIRDSQPSDSATY LCAMSWNSGNTPLVFGKGTRLSVIA (SEQ ID NO: 92) or a sequence with at least 90% identity to SEQ ID NO: 92, and a beta chain variable domain of a sequence of MDTWLVCWAIFSLLKAGLTEPEVTQTPSHQVTQMGQEVILRCVPISNHLYFYWY RQILGQKVEFLVSFYNNEISEKSEIFDDQFSVERPDGSNFTLKIRSTKLEDSAMYFC ASSVTGVRNTIYFGEGSWLTVV (SEQ ID NO: 93) or a sequence with at least 90% identity to SEQ ID NO: 93; or
h) an alpha chain variable domain of a sequence of MTSIRAVFIFLWLQLDLVNGENVEQHPSTLSVQEGDSAVIKCTYSDSASNYFPWY KQELGKRPQLIIDIRSNVGEKKDQRIAVTLNKTAKHFSLHITETQPEDSAVYFCAA YGQKLLFARGTMLKVDL (SEQ ID NO: 94) or a sequence with at least 90% identity to SEQ ID NO: 94, and a beta chain variable domain of a sequence of MGIRLLCRVAFCFLAVGLVDVKVTQSSRYLVKRTGEKVFLECVQDMDHENMFW YRQDPGLGLRLIYFSYDVKMKEKGDIPEGYSVSREKKERFSLILESASTNQTSMY LCASSLLEPDLNTGELFFGEGSRLTVL (SEQ ID NO: 95) or a sequence with at least 90% identity to SEQ ID NO: 95; or
i) an alpha chain variable domain of a sequence of MWGVFLLYVSMKMGGTTGQNIDQPTEMTATEGAIVQINCTYQTSGFNGLFWYQ QHAGEAPTFLSYNVLDGLEEKGRFSSFLSRSKGYSYLLLKELQMKDSASYLCAV NTDKLIFGTGTRLQVFP (SEQ ID NO: 96) or a sequence with at least 90% identity to SEQ ID NO: 96, and a beta chain variable domain of a sequence of MGTSLLCWMALCLLGADHADTGVSQDPRHKITKRGQNVTFRCDPISEHNRLYW YRQTLGQGPEFLTYFQNEAQLEKSRLLSDRFSAERPKGSFSTLEIQRTEQGDSAM YLCASSNPGNSDFGPGTRLTVL (SEQ ID NO: 97) or a sequence with at least 90% identity to SEQ ID NO: 97; or
j) an alpha chain variable domain of a sequence of MAMLLGASVLILWLQPDWVNSQQKNDDQQVKQNSPSLSVQEGRISILNCDYTNS MFDYFLWYKKYPAEGPTFLISISSIKDKNEDGRFTVFLNKSAKHLSLHIVPSQPGD SAVYFCAAVSTGSARQLTFGSGTQLTVLP (SEQ ID NO: 98) or a sequence with at least 90% identity to SEQ ID NO: 98, and a beta chain variable domain of a sequence of MGSRLLCWVLLCLLGAGPVKAGVTQTPRYLIKTRGQQVTLSCSPISGHRSVSWY QQTPGQGLQFLFEYFSETQRNKGNFPGRFSGRQFSNSRSEMNVSTLELGDSALYL CASSLAKGANVLTFGAGSRLTVL (SEQ ID NO: 99) or a sequence with at least 90% identity to SEQ ID NO: 99 or
k) an alpha chain variable domain of a sequence of MKKHLTTFLVILWLYFYRGNGKNQVEQSPQSLIILEGKNCTLQCNYTVSPFSNLR WYKQDTGRGPVSLTIMTFSENTKSNGRYTATLDADTKQSSLHITASQLSDSASYI CVVSAWDPAAGNKLTFGGGTRVLVKP (SEQ ID NO: 100) or a sequence with at least 90% identity to SEQ ID NO: 100, and a beta chain variable domain of a sequence of MSISLLCCAAFPLLWAGPVNAGVTQTPKFRILKIGQSMTLQCTQDMNHNYMYW YRQDPGMGLKLIYYSVGAGITDKGEVPNGYNVSRSTTEDFPLRLELAAPSQTSVY FCASSPGGQGLDTQYFGPGTRLTVL (SEQ ID NO: 101) or a sequence with at least 90% identity to SEQ ID NO: 101; or
l) an alpha chain variable domain of a sequence of MACPGFLWALVISTCLEFSMAQTVTQSQPEMSVQEAETVTLSCTYDTSESDYYLF WYKQPPSRQMILVIRQEAYKQQNATENRFSVNFQKAAKSFSLKISDSQLGDAAM YFCAYNRNDMRFGAGTRLTVKP (SEQ ID NO: 102) or a sequence with at least 90% identity to SEQ ID NO: 102, and a beta chain variable domain of a sequence of MSNQVLCCVVLCFLGANTVDGGITQSPKYLFRKEGQNVTLSCEQNLNHDAMYW YRQDPGQGLRLIYYSQIVNDFQKGDIAEGYSVSREKKESFPLTVTSAQKNPTAFY LCASSTDRDNQPQHFGDGTRLSIL (SEQ ID NO: 103) or a sequence with at least 90% identity to SEQ ID NO: 103;or
m) an alpha chain variable domain of a sequence of MLTASLLRAVIASICVVSSMAQKVTQAQTEISVVEKEDVTLDCVYETRDTTYYLF WYKQPPSGELVFLIRRNSFDEQNEISGRYSWNFQKSTSSFNFTITASQVVDSAVYF CALSEALTSGTYKYIFGTGTRLKVLA (SEQ ID NO: 104) or a sequence with at least 90% identity to SEQ ID NO: 104 and a beta chain variable domain of a sequence of MLLLLLLLGPGISLLLPGSLAGSGLGAVVSQHPSWVICKSGTSVKIECRSLDFQAT TMFWYRQFPKQSLMLMATSNEGSKATYEQGVEKDKFLINHASLTLSTLTVTSAH PEDSSFYICSASVGKSSYEQYVGPGTRLTVT (SEQ ID NO: 105) or a sequence with at least 90% identity to SEQ ID NO: 105.

8. The polypeptide of any of claims 1-7, wherein he TCR comprises an alpha chain TRAC constant domain sequence and/or a beta chain TRBC1 or TRBC2 constant domain sequence.

9. The polypeptide of claim 8, wherein the alpha and or beta constant domain sequence is derived from a mouse TCR.

10. A nucleic acid encoding one or more polypeptide of any of claims 1-9.

11. The nucleic acid of claim 10, wherein the nucleic acid is a DNA or RNA molecule, optionally wherein the DNA is a cDNA or the RNA molecule is an mRNA.

12. A vector comprising the nucleic acid of claim 10 or claim 11.

13. The vector of claim 11, wherein the vector is an expression vector, a plasmid, or a viral vector, optionally wherein the viral vector is a retroviral vector, lentiviral vector or adenoviral vector.

14. A cell or population of cells comprising one or more polypeptide of any of claims 1-9, and/or one or more nucleic acid of any of claim 10 or 11, and/or a vector of any of claims 11-13.

15. The cell or population of cells of claim 14, wherein the cell is a T-cell, or the population of cells is made up of T-cells.

16. The cell or population of cells of claim 15, wherein the T-cell is a CD4+ T-cell or a CD8+ T-cell, or the population of cells is made up of CD4+ T-cells or a CD8+ T-cells or a mixture thereof.

17. A pharmaceutical composition comprising one or more polypeptide of any of claims 1-9, and/or one or more nucleic acid of any of claim 10 or 11, a vector of any of claims 11-13, or a cell or population of cells of any of claims 14-16.

18. A polypeptide of any of claims 1-9, and/or one or more nucleic acid of any of claim 10 or 11, and/or one or more vector of any of claims 11-13, and/or one or more cell or population of cells of any of claims 14-16, and/or one or more pharmaceutical composition of claim 17, for use in treating and/or preventing virus infection in a subject.

19. The polypeptide, nucleic acid, vector, cell or population of cells, or pharmaceutical composition for use of claim 18, wherein the virus infection is HIV-1 infection.

20. The polypeptide, nucleic acid, vector, cell or population of cells, or pharmaceutical composition for use of claim 18 or 19, in combination with one or more further therapeutic agent, optionally wherein the one or more further therapeutic agent comprises or consists of a cytotoxic agent, an agent that activates latent HIV-1 in viral reservoirs, an antibody or receptor mimic that neutralizes or inactivates HIV-1, and/or an immunomodulatory agent, such as IL-2, IFN-gamma, an anti-CD3 antibody.

21. A method of treating or preventing virus infection in a subject, comprising administering to the subject one or more polypeptide of any of claims 1-9, and/or one or more nucleic acid of any of claim 10 or 11, and/or one or more vector of any of claims 11-13, and/or one or more cell or population of cells of any of claims 14-16, and/or one or more pharmaceutical composition of claim 17.

22. The method of claim 21, wherein the virus infection is HIV-1 infection.
